(19) United States
(12) Patent Application Publication
(10) Pub. No.: US 2026/0357271 A1
(43) Pub. Date: Nov. 12, 2026

(54) SYNTHETIC FORMOLASE PATHWAY

(71) Applicant: Technische Universität Wien, Wien (AT)

(72) Inventors: Matthias STEIGER, Wien (AT); Roghayeh SHIRVANI, Wien (AT)

(73) Assignee: Technische Universität Wien, Wien (AT)

(21) Appl. No.: 18/997,063

(22) PCT Filed: Aug. 7, 2023
(86) PCT No.: PCT/EP2023/071821

§ 371 Date: Jan. 18, 2025

(30) Foreign Application Priority Data
Aug. 8, 2022 (AT) A50597/2022

(51) Int. Cl. C12N 15/52; C12N 9/04; C12N 15/81; C12P 7/04; C12P 7/48; C12P 7/52; C12P 7/56; C12P 21/00

(52) U.S. Cl. CPC C12N 15/52; C12N 9/0006; C12N 15/815; C12P 7/04; C12P 7/48; C12P 7/52; C12P 7/56; C12P 21/00; C12Y 101/01244; C12Y 101/03013

(57) ABSTRACT

A eukaryotic cell which is engineered to express a synthetic formolase (FLS) pathway comprising a recombinant FLS to biotransform formaldehyde into dihydroxyacetone.

### FIELD OF THE INVENTION

The invention relates to eukaryotic cells engineered to express a synthetic formolase (FLS) pathway, in particular wherein the FLS pathway is comprised in an organelle of the eukaryotic cell, methods of producing biomass and/or expression products of the eukaryotic cell in a cell culture using a C₁ (one carbon) compound as a carbon source, and fusion proteins comprising an FLS that is fused to a peroxisomal targeting signal (PTS), to target expression of the FLS into the cell's peroxisome.

### BACKGROUND OF THE INVENTION

Methanol plays a crucial role in a future methanol economy. Currently methanol is produced from fossil resources but “green-methanol” or “bio-methanol” can be directly produced from CO₂. As a liquid under ambient conditions, methanol has the advantage to be more easily transported and stored than molecular hydrogen. It is already used as a bulk chemical and multiple processing routes are available. It can serve as fuel and in addition it can be utilized as a carbon source by eukaryotic cells, Ribulose Monophosphate cycle, Dihydroxyacetone cycle, ribulose bisphosphate cycle or the Xylulose Monophosphate cycle. All natural pathways consume high energy (in form of ATP or NAD(P)H). Komagataella phaffii (herein also referred to as Pichia pastoris) is an industrially used methylotrophic yeast and it can grow on methanol as a single carbon source to high biomass densities.

The methanol assimilation pathway (Xylulose-Monophosphate cycle, XuMP) has similarities to the Calvin-Benson-Bassham cycle and the pentose-phosphate pathway. However, this pathway requires at least 10 enzymes to enable the conversion from methanol to dihydroxyacetone (DHA). In addition, three (3) moles of ATP are required for the conversion of three moles of (3) formaldehyde to one (1) mol of Dihydroxyacetone phosphate (DHAP). In order to produce ATP a significant amount of methanol has to be dissimilated by P. pastoris to CO₂ obtaining NADH and thus ATP via the respiratory chain.

Siegel et al. (2015) describe a computationally designed FLS for use in a metabolic pathway using purified enzymes.

US2013196359A1 discloses an in vitro method for converting formaldehyde to dihydroxyacetone by a formolase.

Wang et al. (Bioresour. Bioprocess. 2017, 4:41) describe biological conversion of methanol by evolved Escherichia coli carrying a linear methanol assimilation pathway. The linear pathway consists of two enzymatic reactions: oxidation of methanol into formaldehyde by methanol dehydrogenase and carboligation of formaldehyde into DHA by formolase (FLS). E. coli expressing recombinant FLS could not initiate growth with methanol as the sole carbon source. Methanol assimilation was improved by adaptive evolution to provide for FLS mutants.

Cai et al. (2021) and WO2022042427A1 disclose cell-free chemoenzymatic starch synthesis from carbon dioxide. A mutant of the formolase enzyme (FLS-M3) was described.

CN113151230A discloses FLS mutants.

CN113122525A discloses mutant formaldehyde-converting enzymes and expression of said mutants in a bacterium.

CN110438169A discloses a method for catalytic synthesis of 1-hydroxy-2-butanone in bacterial cells using formaldehyde and propionaldehyde as raw materials and an engineered E. coli strain containing formaldehyde lyase (FLS).

Van der Klei et al. (2006) review the role of peroxisomes in methanol metabolism of methylotrophic yeasts.

CN107475281A discloses E. coli engineered to comprise a methanol metabolism pathway including methanol dehydrogenase and formaldehyde lyase FLS.

US20200181629A1 discloses a yeast comprising a synthetic Calvin cycle wherein a ribulose-bisphosphate carboxylase (RuBisCO) gene and ribulose phosphate kinase (PRK) gene are fused with a peroxisomal targeting signal (PTS).

Gassler et al. (2020) describe a Pichia pastoris strain which is an autotroph capable of growth on CO₂. A CO₂ assimilation pathway was engineered into P. pastoris when targeted to the peroxisome of the yeast.

RuBmayer et al. (2015) describe systems-level organization of yeast methylotrophic lifestyle. The authors analyzed the regulation patterns of 5,354 genes, 575 proteins, 141 metabolites, and fluxes through 39 reactions of P. pastoris comparing growth on glucose and on a methanol/glycerol mixed medium, respectively. It was found that the entire methanol assimilation pathway is localized to peroxisomes rather than employing part of the cytosolic pentose phosphate pathway for xylulose-5-phosphate regeneration.

KR20210038805A discloses methylotrophic bacteria with enhanced 1,2-propylene glycol (propanediol; PDO) productivity and a 1,2-PDO production method using the same.

WO2012037413A2 discloses systems, compounds and methods for the conversion of C1 carbon compounds to higher carbon compounds useful for the generation of commodity compounds.

WO2012170292A1 discloses modulation of metabolic pathways for improving bioprocess performance and secreted protein productivity of yeast. An isolated fungal host cell, such as Pichia pastoris, lacking full wild-type levels of dihydroxyacetone synthase activity due to a knocked-out DAS1 or DAS2, is provided. The host cell further comprises a heterologous polynucleotide encoding a heterologous polypeptide. Methods for expressing the heterologous polypeptide in such cells, and purifying the expressed polypeptide are also disclosed.

Novel strategies are needed to address current challenges in energy storage and carbon sequestration. In particular, there is a need for new biosynthesis methods to produce biomass or expression products in a eukaryotic cell culture.

### SUMMARY OF THE INVENTION

It is the objective to provide a new metabolic pathway for carbon fixation in eukaryotic cells. It is a particular objective to provide for cell cultures capable of using a C₁ compound as a carbon source to effectively produce biomass or expression products including e.g., recombinant proteins or cellular metabolites. It is a further objective to engineer cells for producing higher yields of biomass or expression products.

The objective is solved by the subject matter as claimed and as further described herein.

The invention provides for a eukaryotic cell which is engineered to express a synthetic formolase (FLS) pathway comprising a recombinant FLS to biotransform formaldehyde into dihydroxyacetone (DHA).

DHA is herein understood to refer to DHA in its unphosphorylated form or its phosphorylated form, dihydroxyacetone phosphate (DHAP). DHA is also known as 1,3-dihydroxypropan-2-one, 1,3-dihydroxypropanone, or glycerone.

Specifically, the biotransformation comprises biosynthesis of dihydroxyacetone from formaldehyde through enzymatic reaction with the FLS which converts formaldehyde to dihydroxyacetone.

Specifically, the FLS is a lyase which catalyzes the carboligation of three one-carbon formaldehyde molecules into one three-carbon dihydroxyacetone molecule. Herein described is the recombinant expression of an FLS gene that was introduced into eukaryotic cells, in particular into peroxisomes of a methylotrophic yeast, to replace the natural Xylulose Monophosphate pathway of Pichia pastoris (Komagataella phaffi). To achieve targeting to the peroxisomes, a peroxisome targeting signal (PTS) was fused to the FLS. Cells containing this pathway were able to grow on methanol as carbon source.

It was found that a eukaryotic cell that expresses a recombinant FLS comprises the new FLS pathway. Specifically, the new FLS pathway comprises assimilating a C₁ compound like methanol to produce formaldehyde in an enzymatic reaction prior to the FLS enzymatic reaction. Therefore, the invention provides for engineered cells comprising a new carbon fixation pathway including the FLS pathway, which has the advantage of only a small number of thermodynamically favorable chemical transformations that convert a C₁ compound into a three-carbon sugar in central metabolism.

The new FLS pathway has a lower energy consumption compared to the natural pathway. By the FLS pathway described herein, dihydroxyacetone phosphate can be produced in an energy efficient way, since the formose reaction catalyzed by the enzyme formolase (FLS) can directly form DHA from three (3) moles of formaldehyde. Only one (1) mol of ATP is required (instead of three mol ATP as in the Xylulose-Monophosphate cycle) to form dihydroxyacetone phosphate, which is an intermediate of glycolysis and thus of the central carbon metabolism.

Herein described are also recombinant FLS constructs (proteins and nucleic acid molecules) which can be expressed in eukaryotic cells, in particular in cellular compartments like organelles or peroxisomes, and respective expression cassettes.

According to a specific aspect, the FLS pathway comprises or consists of the recombinant FLS to convert formaldehyde into dihydroxyacetone (DHA).

Specifically, recombinant FLS is expressed under the control of a methanol-inducible promoter, preferably pDAS2 or pAOX1.

Specifically, the invention provides for a yeast cell comprising a peroxisome, wherein the yeast cell is engineered to express a synthetic formolase (FLS) pathway in the peroxisome, comprising a recombinant FLS to biotransform formaldehyde into dihydroxyacetone (DHA), wherein the FLS is expressed as an FLS fusion protein that comprises the FLS fused to a peroxisomal targeting signal (PTS).

Specifically, the FLS pathway further comprises one or more enzymes to biotransform a C₁ compound into formaldehyde, preferably wherein the C₁ compound is any one of methanol, formate, methane, carbon monoxide, or carbon dioxide.

According to a specific aspect, the FLS pathway further comprises a methanol to formaldehyde converting enzyme (“MFCE”), such as an alcohol oxidase or a methanol dehydrogenase, to biotransform methanol into formaldehyde. Specifically, the alcohol oxidase is a methanol oxidase.

Specifically, the FLS pathway is a linear pathway.

Specifically, the FLS pathway is comprised in a metabolic pathway which further comprises one or more enzymes of gluconeogenesis using DHA (or DHAP) as a precursor.

According to a specific aspect, the FLS pathway is comprised in an organelle of the cell, preferably a peroxisome or mitochondria.

Specifically, the FLS is a heterologous protein, in particular an artificial enzyme originating from or comprising a wild-type (such as naturally-occurring in any organism) or a mutant FLS. Optionally, the FLS is fused to one or more heterologous sequences.

Specifically, the FLS is expressed as an FLS fusion protein that comprises the FLS fused to a peroxisomal targeting signal (PTS), preferably wherein the PTS is selected from the group consisting of the Minimal PTS1 sequence (“SKL”), the “PTS1 consensus sequence” (SEQ ID NO:17), the “PTS2 consensus sequence” (SEQ ID NO:18), SEQ ID NO:19, 20, 21, 22, 23, 24, 25, and 26.

Specifically, the PTS comprises or consists of an amino acid sequence selected from the group consisting of SKL, SEQ ID NO:17, 18, 19, 20, 21, 22, 23, 24, 25, and 26.

SEQ ID NO:17 comprises variables such that the amino acid sequence can have a length of 3-12 amino acids. The respective embodiments of SEQ ID NO:17 are provided as SEQ ID NO:35-44.

Specifically, the PTS can be fused at the C-terminus or the N-terminus of the FLS amino acid sequence, with or without a linking sequence.

Specifically, the PTS1 sequence comprises or consists of the sequence motif of a PTS1 sequence having a consensus sequence of (S/A/C/T)-(K/R/H)-(L/M/F) (i.e., the “PTS1 consensus sequence”), which is optionally extended at the N-terminus by one or more, up to nine amino acids, (X)₀₋₉. Therefore, the PTS1 consensus sequence is characterized by the following formula: (X)₀₋₉-(S/A/C/T)-(K/R/H)-(L/M/F) (see SEQ ID NO:17).

Preferably a PTS of the PTS1 consensus sequence is fused to the C-terminus.

Specifically, the PTS2 sequence comprises or consists of the sequence motif of a PTS2 sequence having a consensus sequence of (R/K)-(L/1/V)-(X)₅-(H/Q)-(L/A) (i.e., the “PTS2 consensus sequence”, see SEQ ID NO:18), which is preferably fused to the N-terminus.

In the consensus sequences, the “forward slash” indicates alternative amino acids in the sequence, and the “hyphen” indicates a peptidic linkage.

Specifically, the FLS pathway is expressed into the cell's peroxisome, in particular wherein the cell is a yeast, such as e.g., a methylotrophic yeast. Preferably, the FLS pathway expressed into the peroxisome comprises an MFCE and a FLS to convert methanol to dihydroxyacetone.

According to a specific aspect, the FLS pathway comprises an alcohol oxidase and FLS, wherein:
a) the MFCE is an endogenously expressed peroxisomal enzyme; and
b) the FLS is an enzyme that is targeted to the peroxisome by fusing a PTS.

Specifically, the FLS is characterized by one or more of the following features:
a) the FLS is a bacterial FLS or a benzaldehyde lyase of bacterial origin, such as originating from Pseudomonas fluorescens e.g., Pseudomonas fluorescens biovar I, preferably wherein the FLS is identified as SEQ ID NO:33 or by the NCBI accession number: 2AGO_A (Chain A, benzaldehyde lyase), or a naturally-occurring FLS homolog or ortholog thereof;
b) the FLS is a recombinant FLS which is a mutant of a), preferably wherein the recombinant FLS comprises SEQ ID NO:33 which is engineered to incorporate one or more point mutations, preferably up to 15, 14, 13, 12, 11, 10, 9, 8, or 7 point mutations, wherein the point mutations comprise any one or more point mutations, preferably comprising
   i) at least 1, 2, 3, 4, 5, 6, or 7 (all) of any of the following mutations: A281, W89R, L90T, R188H, A394G, G419N, or A480W; or
   ii) at least 1, 2, 3, 4, 5, 6, or 7 (all) of any of the following mutations: A28L, W89R, R188H, N283H, A394G, G419N, and A480W; or
   iii) at least 1, 2, 3, 4, or 5 of any of the following mutations: W89R, R188H, A394G, G419N, or A480W, and optionally one, two or three additional mutations selected from: A281, A28L, L90T, N283H;
c) the FLS comprises or consists of SEQ ID NO:31 or SEQ ID NO:32;
d) the FLS comprises at least any one of 90%, 95%, 96%, 97%, 98%, 99% or 100% sequence identity to any one of the FLS of a), b), or c);
e) the FLS is functional as determined in a colorimetric enzymatic assay, such as described herein;
f) the nucleotide sequence encoding the FLS is codon-optimized for expression in the cell.

Specific FLS described herein are:
a) a wild-type FLS of Pseudomonas fluorescens, wherein the FLS is identified as SEQ ID NO:33 or by the NCBI accession number: 2AGO_A (Chain A, benzaldehyde lyase);
b) a recombinant FLS which comprises or consists of SEQ ID NO:32, which recombinant FLS comprises the point mutations A281, W89R, L90T, R188H, A394G, G419N, and A480W as compared to SEQ ID NO:33;
c) a recombinant FLS which comprises or consists of SEQ ID NO:31, which recombinant FLS comprises the point mutations A28L, W89R, R188H, N283H, A394G, G419N, and A480W as compared to SEQ ID NO:33.

The FLS identified as SEQ ID NO:31 or SEQ ID NO:32 is a functional FLS mutant of a wild-type FLS.

The wild-type FLS identified by SEQ ID NO:33 comprises an amino acid (aa) sequence of 563 aa length. The mutant FLS can be longer than SEQ ID NO:33 e.g., by a C-terminal extension of the aa sequence. For example, the C-terminal extension of SEQ ID NO:33 can be an extension of 1 to 12 consecutive aa of “GSTENLYFQSGA” (SEQ ID NO:34) wherein the extension starts with the first aa indicated in SEQ ID NO:34.

It is understood that a recombinant or mutant FLS originating from a wild-type FLS such as comprising or consisting of SEQ ID NO:33, may or may not comprise such C-terminal extension. In particular, it is understood that any of the recombinant or mutant FLS comprising or consisting of SEQ ID NO:31 or SEQ ID NO:32, or a functional variant of any of the foregoing, may or may not comprise such C-terminal extension.

SEQ ID NO:31 or SEQ ID NO:32 comprises an aa sequence of 575 aa length.

Specifically, the FLS comprises of consists of SEQ ID NO:31 or SEQ ID NO:32, or a functional variant of any of the foregoing, preferably wherein
a) the functional variant of SEQ ID NO:31 comprises at least 1, 2, 3, 4, 5, 6, or 7 (all) of the point mutations A28L, W89R, R188H, N283H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:31;
b) the functional variant of SEQ ID NO:32 comprises at least 1, 2, 3, 4, 5, 6, or 7 (all) of the point mutations A281, W89R, L90T, R188H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:32.

According to a specific aspect, the cell is a microbial cell, a mammalian, insect, plant or algae cell.

Specifically, the cell is:
a) a yeast cell, preferably of a genus selected from the group consisting of Pichia, Komagataella, Hansenula, Saccharomyces, Kluyveromyces, Candida, Ogataea, Yarrowia, and Geotrichum, preferably Pichia pastoris, Komagataella phaffii, Komagataella pastoris, Komagataella pseudopastoris, Saccharomyces cerevisiae, Ogataea minuta, Kluyveromces lactis, Kluyveromyes marxianus, Yarrowia lipolytica or Hansenula polymorpha, preferably a methylotrophic yeast;
b) a cell of filamentous fungi, preferably of a genus selected from the group consisting of Aspergillus, Penicillium, Trichoderma, Neurospora, Rhizopus preferably Aspergillus niger, Aspergillus awamori Aspergillus terreus, Aspergillus oryzae, Aspergillus nidulans, Neurospora crassa, Rhizopus oryzae, Penicillium chrysogenum or Trichoderma reesei;
c) a non-human primate, human, rodent, or bovine cell, preferably a mouse myeloma (NSO)-cell line, a Chinese hamster ovary (CHO)-cell line, HT1080, H9, HepG2, MCF7, MDBK Jurkat, MDCK, NIH3T3, PC12, BHK (baby hamster kidney cell), VERO, SP2/0, YB2/0, YO, C127, L cell, COS, QC1-3, HEK-293, PER.C6, HeLA, EBI, EB2, EB3, oncolytic or hybridoma-cell line;
d) an insect cell, preferably a Sf9, Mimic™ Sf9, Sf21, High Five (BT1-TN-5B1-4), or BT1-Ea88 cell;
e) an algae cell, preferably of the genus Amphora, Bacillariophyceae, Dunaliella, Chlorella, Chlamydomonas, Cyanophyta (cyanobacteria), Nannochloropsis, Spirulina, or Ochromonas); or
f) a plant cell, preferably a cell from monocotyledonous plants, preferably maize, rice, wheat, or Setaria, or from a dicotyledonous plant, preferably cassava, potato, soybean, tomato, tobacco, alfalfa, Physcomitrella patens or Arabidopsis.

The preferred yeast host cells are derived from methylotrophic yeast, such as from Pichia or Komagataella e.g., Pichia pastoris, or Komagataella pastoris, or K. phaffii, or K. pseudopastoris. Examples of the host include yeasts such as P. pastoris. Examples of P. pastoris strains include CBS 704 (=NRRL Y-1603=DSMZ 70382), CBS 2612 (=NRRL Y-7556), CBS 7435 (=NRRL Y-11430), CBS 9173-9189 (CBS strains: CBS-KNAW Fungal Biodiversity Centre, Centraalbureau voor Schimmelcultures, Utrecht, The Netherlands), and DSMZ 70877 (German Collection of Microorganisms and Cell Cultures), but also strains from Invitrogen, such as X-33, GS115, KM71 and SMD1168. Examples of S. cerevisiae strains include W303, CEN.PK and the BY-series (EUROSCARF collection). All of the strains described above have been successfully used to produce transformants and express heterologous genes.

A preferred yeast host cell may be a P. pastoris or S. cerevisiae host cell, specifically wherein such host cell contains heterologous or recombinant promoter sequences, which may be derived from a P. pastoris or S. cerevisiae strain, different from the production host. In another specific embodiment the host cell described herein comprises a recombinant expression construct described herein comprising the promoter originating from the same genus, species or strain as the host cell.

According to a specific aspect, the cell is an engineered Mut-yeast, specifically, wherein the Mut-yeast is characterized by a phenotype with reduced growth on methanol.

Specifically, the Mut-yeast is engineered to reduce expression of a dihydroxyacetone synthase (DAS) compared to an endogenous expression thereof, in particular as compared to endogenous expression in the yeast without such engineering, preferably wherein said engineering is a deletion, knock-out or disruption of any one or both of the DAS1 and DAS2 genes.

According to a specific aspect, the cell can be cultured in a cell culture using a cell culture medium comprising a C₁ compound as a carbon source, in particular wherein the carbon source is used as a source of energy and/or as a source of producing fermentation products including e.g., expression products.

Specifically, the cell is capable of utilizing a C₁ compound as the sole carbon source for producing biomass and/or an expression product, such as e.g., in a cell culture.

The invention further provides for a method of culturing the cell described herein in a cell culture using a C₁ compound as a carbon source, thereby obtaining biomass and/or an expression product in the cell culture, preferably wherein the C₁ compound is the sole carbon source.

Specifically, the expression product is a protein of interest (POI) or a metabolite, preferably wherein:
a) the POI is a peptide or protein selected from the group consisting of an antigen-binding protein, a therapeutic protein, an enzyme, a peptide, a protein antibiotic, a toxin fusion protein, a carbohydrate-protein conjugate, a structural protein, a regulatory protein, a vaccine antigen, a growth factor, a hormone, a cytokine, a process enzyme;
b) the metabolite is an amino acid, organic acid, alcohol, sugar alcohol, carbohydrate, vitamin, amine, aldehyde, ketone, or a polyhydroxyketone, preferably wherein the metabolite is selected from the group consisting of lactic acid, citric acid, propionic acid, butyric acid, valeric acid, hexanoic acid, adipic acid, succinic acid, fumaric acid, malic acid, 2,5-furan dicarboxylic acid, aspartic acid, glucaric acid, gluconic acid, glutamic acid, itaconic acid, levulinic acid, acrylic acid, 3-hydroxy propionic acid, ethanol, propanol, isopropanol, butanol, pentanol, hexanol, heptanol, octanol, butanediol, 1,3-propanediol, 1,2-propanediol, 2-amino-1,3-propanediol, 3-hydroxybutyrate, poly-3-hydroxybutyrate, 3-hydroxy propionaldehyde, 3-hydroxybutyrolactone, xylitol, arabinitol, sorbitol, mannitol, vitamin C, riboflavin, thiamine, tocopherol, cobalamin, pantothenic acid, biotin, pyridoxine, niacin, folic acid, diamino pentane, diamino hexane and dihydroxyacetone.

Specifically, for the purpose of expressing an expression product, a recombinant expression cassette is used, which comprises one or more heterologous nucleic acid sequence(s).

According to a preferred aspect, the cell is a yest such as e.g., a methylotrophic yeast, and the expression cassette comprises a heterologous coding sequence (or gene) which is operably linked to a methanol-inducible promoter.

According to a specific aspect, a method of producing biomass and/or an expression product in a cell culture is provided, using a cell described herein and a C₁ compound, preferably wherein the C₁ compound is the sole carbon source in the cell culture.

The methods described herein employ the cell as described herein. Therefore, the methods described herein are specifically characterized by the features of the cell as described herein.

The invention further provides for a fusion protein comprising a formolase (FLS) that is fused to a peroxisomal targeting signal (PTS).

Specifically, the fusion protein is characterized by the features described as features of the FLS as used in the FLS pathway described herein.

Specifically, the fusion protein is characterized by one or more of the following features:
a) the PTS is selected from the group consisting of the Minimal PTS1 sequence (“SKL”), the “PTS1 consensus sequence” (as defined herein), the “PTS2 consensus sequence” (SEQ ID NO:17), SEQ ID NO:18, 19, 20, 21, 22, 23, 24, 25, and 26; and
b) the FLS is as further described herein.

According to a specific aspect, the fusion protein comprises an FLS which is a recombinant FLS such as a functional FLS mutant of a wild-type bacterial FLS, such as an FLS of bacterial origin e.g., originating from Pseudomonas fluorescens, in particular Pseudomonas fluorescens biovar I, wherein the recombinant FLS comprises the amino acid sequence SEQ ID NO:33 wherein the amino acid sequence is engineered to incorporate one or more point mutations, preferably up to 15, 14, 13, 12, 11, 10, 9, 8, or 7 point mutations, wherein the point mutations comprise any one or more point mutations, preferably comprising
i) at least 1, 2, 3, 4, 5, 6, or 7 (all) of any of the following mutations: A281, W89R, L90T, R188H, A394G, G419N, or A480W; or
ii) at least 1, 2, 3, 4, 5, 6, or 7 (all) of any of the following mutations: A28L, W89R, R188H, N283H, A394G, G419N, and A480W; or
iii) at least 1, 2, 3, 4, or 5 of any of the following mutations: W89R, R188H, A394G, G419N, or A480W, and optionally one, two or three additional mutations selected from: A281, A28L, L90T, N283H;
   preferably wherein the recombinant FLS comprises or consists of SEQ ID NO:31 or SEQ ID NO:32.

Specifically, the fusion protein comprises an FLS which comprises of consists of SEQ ID NO:31 or SEQ ID NO:32, or a functional variant of any of the foregoing, preferably wherein
a) the functional variant of SEQ ID NO:31 comprises at least 1, 2, 3, 4, 5, 6, or 7 (all) of the point mutations A28L, W89R, R188H, N283H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:31;
b) the functional variant of SEQ ID NO:32 comprises at least 1, 2, 3, 4, 5, 6, or 7 (all) of the point mutations A281, W89R, L90T, R188H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:32.

The invention further provides for a nucleic acid molecule encoding the fusion protein described herein, preferably wherein the nucleic acid molecule is codon-optimized for expression in a eukaryotic host cell.

Specifically, the nucleic acid molecule is an isolated nucleic acid molecule. The isolated nucleic acid molecule may be incorporated into an expression cassette or an expression construct comprising the expression cassette, such as a vector, plasmid, or artificial chromosome. A preferred yeast expression vector is for expression in a yeast host cell, such as selected from the group consisting of methylotrophic yeasts represented by the genera Ogataea, Hansenula, Pichia, Candida and Torulopsis. Specifically, plasmids derived from pPICZ, pGAPZ, pPIC9, pPICZalfa, pGAPZalfa, pPIC9K, pGAPHis or pPUZZLE can be used as a vector.

The invention further provides for an expression cassette comprising the nucleic acid molecule described herein and regulatory sequences to express the fusion protein in a eukaryotic host cell.

Specifically, the FLS fusion protein is expressed under the control of a methanol-inducible promoter, preferably pDAS2 or pAOX1.

Specifically provided herein is a eukaryotic cell comprising the expression cassette described herein.

Specifically, the recombinant FLS described herein is a functional enzyme, wherein the enzymatic activity is determined by a standard colorimetric assay such as described in Example 4, or by Cai et al. (Cai et al. 2021), or by Siegel et al. (Siegel et al. 2015).

Specifically, a FLS is considered functional if a catalytic efficiency higher than 0.01 M⁻¹*s⁻¹ can be detected.

### FIGURES

FIG. 1: Schematic representation of the constructed and integrated gene cassettes for generation of the P. pastoris strain (A) ΔDAS1, ΔDAS2, ΔAOX1, (pDAS2::FLS::HpHr)+ and (B) strain ΔDAS1, ΔDAS2, ΔAOX1, (pAOX1::FLS::G418^{r}).

FIG. 2: Confirmation of the FLS gene expression cassettes by PCR amplification using genomic DNA as template. Amplified gene fragments (1-6) SEQ3 from ΔDAS1, ΔDAS2, ΔAOX1, (pDAS2::FLS::HpH^{r})⁺, transformants, and (7-12) SEQ4 from ΔDAS1, ΔDAS2, ΔAOX1, (pAOX1::FLS::G418^{r})⁺, (M) Quick-Load 1 kb Plus DNA Ladder (New England Biolabs).

FIG. 3: (A) The growth curves of the FLS expressing P. pastoris transformants compared to the parental strain and an empty vector strain in synthetic M2 media with methanol as carbon source. (B) The logarithmic representation of the same growth curves presenting the linear fitted regions used to calculate the growth rate.

FIG. 4: Microscopic images after 40 hours of incubation in M2/methanol media (A) FLS expressing P. pastoris strain ΔDAS1, ΔDAS2, ΔAOX1, (pAOX1::FLS::G418r)+ and of (B) the parental P. pastoris MUT⁻ strain ΔDAS1, ΔDAS2, ΔAOX1 at a 1000× magnification with a total field of view diameter of 200 μm.

FIG. 5:

SEQ ID NO:1: DNA sequence of FLS::PTS1 coding sequence

SEQ ID NO:2: Protein sequence of FLS::PTS1 (fusion protein)

SEQ ID NO:3: pDAS2::FLS::HpH^{r} cassette sequenced integrated into genome of

P. pastoris CBS7435ΔDAS1, ΔDAS2, ΔAOX1

SEQ ID NO:4: pAOX1::FLS::HpH^{r} cassette sequenced integrated into genome of

P. pastoris CBS7435ΔDAS1, ΔDAS2, ΔAOX1

SEQ ID NO:5: PCR Primer Amplification DAS1 locus, (DAS1-F)

SEQ ID NO:6: PCR Primer Amplification DAS1 locus, (DAS1-R)

SEQ ID NO:7: PCR Primer Amplification DAS2 locus, (DAS2-F)

SEQ ID NO:8: PCR Primer Amplification DAS2 locus, (DAS2-R)

SEQ ID NO:9: PCR Primer Amplification AOX1 locus, (AOX1-F)

SEQ ID NO:10: PCR Primer Amplification AOX1 locus, (AOX1-R)

SEQ ID NO:11: PCR Screening Primer for FLS cassette integration (Screening PCR-F)

SEQ ID NO:12: PCR Screening Primer for FLS cassette integration (Screening PCR-R)

SEQ ID NO:13: DNA sequence of genomic locus of ΔAOX1 of P. pastoris CBS7435 ΔDAS1, ΔDAS2, ΔAOX1

SEQ ID NO:14: DNA sequence of genomic locus of ΔDAS1 of P. pastoris CBS7435 ΔDAS1, ΔDAS2, ΔAOX1

SEQ ID NO:15: DNA sequence of genomic locus of ΔDAS2 of P. pastoris CBS7435 ΔDAS1, ΔDAS2, ΔAOX1

SEQ ID NO:16: Fusion protein comprising a FLS (from Siegel et al. 2015) and a PTS

Minimal PTS1 sequence: SKL

SEQ ID NO:17: PTS1 consensus sequence: (X)₀₋₉-(S/A/C/T)-(K/R/H)-(L/M/F)

SEQ ID NO:18: PTS2 consensus sequence: (R/K)-(L/1/V)-(X)₅-(H/Q)-(L/A)

SEQ ID NO:19: PTS1 from AOX1 (Pichia pastoris)

SEQ ID NO:20: PTS1 from AOX2 (Pichia pastoris)

SEQ ID NO:21: PTS1 from FBA1 (Pichiapastoris)

SEQ ID NO:22: PTS1 from RKI1-2 (Pichia pastoris)

SEQ ID NO:23: PTS1 from TAL1-2 (Pichia pastoris)

SEQ ID NO:24: PTS1 from CTA1 (Pichia pastoris)

SEQ ID NO:25: PTS2 from POX3 (Saccharomyces cerevisiae)

SEQ ID NO:26: PTS2 from AMO (Hansenula polymorpha)

SEQ ID NO:27: AOX1 from Komagataella phaffii strain ATCC 76273/CBS 7435

SEQ ID NO:28: AOX2 from Komagataella phaffii strain ATCC 76273/CBS 7435,

SEQ ID NO:29: AOD1 from Candida boidinii

SEQ ID NO:30: MOX Hansenula polymorpha

SEQ ID NO:31: recombinant FLS of SEQ ID NO:2

SEQ ID NO:32: recombinant FLS of SEQ ID NO:16

SEQ ID NO:33: FLS of Pseudomonas fluorescens, NCBI accession number: 2AG0_A (Chain A, benzaldehyde lyase)

SEQ ID NO:34: exemplary C-terminal extension, to extend SEQ ID NO:33

SEQ ID NO:35-SEQ ID NO:44: embodiments of SEQ ID NO:17

### DETAILED DESCRIPTION OF THE INVENTION

Unless indicated or defined otherwise, all terms used herein have their usual meaning in the art, which will be clear to the skilled person. Reference is for example made to the standard handbooks. Genetic modifications described herein may employ tools, methods and techniques known in the art, such as described by J. Sambrook et al., Molecular Cloning: A Laboratory Manual (3^{rd} edition), Cold Spring Harbor Laboratory, Cold Spring Harbor Laboratory Press, New York (2001), Lewin “Genes “V”, Oxford University Press, New York (1990), and Janeway et al. “Immunobiology” (5^{th} Ed., or more recent editions), Garland Science, New York (2001).

The terms “comprise”, “contain”, “have” and “include” as used herein can be used synonymously and shall be understood as an open definition, allowing further members or parts or elements. “Consisting” is considered as a closest definition without further elements of the “consisting” definition feature. Thus “comprising” is broader and contains the “consisting” definition.

The term “about” as used herein refers to the same value or a value differing by +/−10% or +/−5% of the given value.

The subject matter of the claims specifically refers to artificial products or methods employing or producing such artificial products, which may be variants of native (wild-type) products. Though there can be a certain degree of sequence identity to the native structure, it is well understood that the materials, methods and uses of the invention, e.g., specifically referring to isolated nucleic acid sequences, amino acid sequences, fusion constructs, expression constructs, engineered cells such as host cells incorporating recombinant expression constructs or synthetic metabolic pathways, and modified proteins, are “man-made” or synthetic, and are therefore not considered as a result of “laws of nature”.

Specific terms as used throughout the specification have the following meaning:

The term “biomass” as used herein refers to the substance obtained by isolating whole cells, fractions or extracts thereof which are obtained from a cell culture, in particular where the biomass includes carbohydrates and/or proteins.

The term “carbon source” is herein understood as “carbon substrate” and shall mean a fermentable carbon substrate which can be used to produce organic carbon compounds, suitable as an energy source for microorganisms. C₁ compounds can be used as a carbon source, which are inorganic or organic compounds that comprise only one carbon atom per molecule or ion. Exemplary C₁ carbon molecules used as substrates for biomass production and other fermentation processes described herein include methane, carbon dioxide (in the gaseous or solubilized form), carbon monoxide, methanol and synthesis gas (a mixture of carbon monoxide and hydrogen), but may also be formate, or mixtures of any of the foregoing. A compound may be used as a sole carbon source, or else a mixture of different compounds can be used as a carbon source.

The term “cell” with respect to an engineered cell as used herein, which comprises a metabolic pathway, which is herein also referred to as a “host cell”, shall refer to a single cell, a single cell clone, or a cell line.

The term “cell line” as used herein refers to an established clone of a particular cell type that has acquired the ability to proliferate over a prolonged period of time. A cell line is typically used for expressing an endogenous or recombinant nucleic acid molecule or gene, a metabolic pathway or products of a metabolic pathway, to produce fermentation products, such as biomass or expression products e.g., polypeptides (including e.g., a protein of interest, POI), nucleic acid products (for example DNA or RNA), cells and/or viruses, or cell metabolites.

A “production host cell line” or “production cell line” is commonly understood to be a cell line ready-to-use for cell culture in a bioreactor to obtain fermentation products.

It is well understood that the term “host cell” refers to recombinant host cells and does not include human beings. Specifically, recombinant host cells as described herein are artificial organisms and derivatives of native (wild-type) host cells.

Specific embodiments described herein refer to a host cell which is engineered as described herein, such as to incorporate and to express a FLS pathway as described herein. Such recombinant host cells are provided as isolated host cell and respective host cell lines or cultures. Recombinant host cells can be cultured ex vivo to produce fermentation products.

The term “cell culture” or “culturing” as used herein with respect to a host cell refers to the maintenance of cells in an artificial, e.g., an in vitro environment, under conditions favoring growth, differentiation or continued viability, in an active or quiescent state, of the cells, specifically in a controlled bioreactor according to methods known in the industry. When culturing a cell culture using appropriate culture media, the cells are brought into contact with the media in a culture vessel or with substrate under conditions suitable to support culturing the cells in the cell culture. Standard cell culture media and techniques are well-known in the art.

Recombinant proteins or cellular metabolites can be produced using the host cell and respective cell lines described herein, by culturing in an appropriate medium, isolating the expressed product or metabolite from the culture, and optionally purifying it by a suitable method.

The host cell described herein can be tested for its capacity to produce expression products by any of the following tests: ELISA, activity assay, capillary electrophoresis, HPLC, or other suitable tests, such as SDS-PAGE and Western Blotting techniques, or mass spectrometry.

The term “expression” as used herein refers to expression of a polynucleotide or gene from an expression cassette, or to the expression of the respective polypeptide or protein.

The term “expression cassette” is herein understood to refer to nucleic acid molecules (herein also referred to as polynucleotides), which contain a desired coding sequence (herein referred to as a gene), and control sequences in operable linkage, so that hosts transformed or transfected with these molecules incorporate the respective sequences and are capable of producing the expression products.

An expression cassette may comprise one or more nucleic acid molecules (e.g., a gene) that are endogenous or heterologous to a host cell. Specifically, the expression cassette may comprise polynucleotides encoding a metabolic pathway, such as a pathway comprising the FLS pathway described herein. Specifically, the metabolic pathway described herein may be encoded by polynucleotides comprised in one or more expression cassettes, e.g., in one or more expression constructs comprised in the host cell.

An expression cassette can be engineered ex vivo or in vivo. For example, an expression cassette can be engineered as part of a vector or an artificial expression construct that can be provided as isolated expression construct that is engineered using in vitro techniques, and optionally incorporated in a cell culture.

Specifically, the recombinant FLS expression cassette described herein comprises the FLS encoding nucleic acid molecule operably linked to one or more regulatory elements that control expression of the FLS.

Specifically, the expression cassette expressing the recombinant FLS described herein comprises one or more regulatory elements not natively associated with the gene encoding the FLS. Such expression cassettes comprising one or more elements that are not natively associated with a gene, are herein understood as artificial or recombinant expression cassettes.

An element of an expression cassette that is “not natively associated with” such gene is typically not operably linked to such gene in a wild-type host cell, whereas the wild-type host cell is endogenously expressing such gene with regulatory elements that are “natively associated with” such gene. A wild-type host cell is herein understood to be a naturally-occurring host cell that is not recombined by any artificial means.

One or more expression cassettes are herein also understood as “expression system”. The expression system may be included in an expression construct, such as an artificial heterologous expression cassette, a vector and in particular a plasmid. The relevant DNA of an expression cassette or construct may also be integrated into a host cell chromosome. Expression may refer to secreted or non-secreted expression products, including polypeptides or metabolites.

Expression cassettes are conveniently provided as expression constructs e.g., in the form of “vectors” or “plasmids”, which are typically DNA sequences that are required for the transcription of cloned recombinant nucleotide sequences i.e., of recombinant genes and the translation of their mRNA in a suitable host organism. Expression vectors or plasmids usually comprise an origin for autonomous replication or a locus for genome integration in the host cells, selectable markers (e.g., an amino acid synthesis gene or a gene conferring resistance to antibiotics such as zeocin, kanamycin, G418 or hygromycin, nourseothricin), a number of restriction enzyme cleavage sites, a suitable promoter sequence and a transcription terminator, which components are operably linked together. The terms “plasmid” and “vector” as used herein include autonomously replicating nucleotide sequences as well as genome integrating nucleotide sequences, such as artificial chromosomes e.g., a bacterial artificial chromosome (BAC) or yeast artificial chromosome (YAC).

Expression vectors may include but are not limited to cloning vectors, modified cloning vectors and specifically designed plasmids. Preferred expression vectors described herein are expression vectors suitable for expressing a recombinant gene in a eukaryotic host cell and are selected depending on the host organism. Appropriate expression vectors typically comprise regulatory sequences suitable for expressing DNA encoding polypeptide or protein of interest in a eukaryotic host cell. Examples of regulatory sequences include promoter, operators, enhancers, ribosomal binding sites, and sequences that control transcription and translation initiation and termination. The regulatory sequences are typically operably linked to the DNA sequence to be expressed.

To allow expression of a recombinant nucleotide sequence in a host cell, a promoter sequence is typically regulating and initiating transcription of the downstream nucleotide sequence, with which it is operably linked. An expression cassette or vector typically comprises a promoter nucleotide sequence which is adjacent to the 5′ end of a coding sequence, e.g., upstream from and adjacent to the coding sequence (e.g., gene of interest) or if a signal or leader sequence is used, upstream from and adjacent to said signal and leader sequence, respectively, to facilitate translation initiation and expression of coding sequences to obtain the expression product.

As used herein, the term “adjacent” is meant to refer to a distance between elements of a nucleic acid sequence which is “within about 10 bp” or “within about 5 bp” of the nucleic acid sequence.

Specific expression cassettes described herein comprise a promoter operably linked to a coding nucleotide sequence. Specifically, the promoter can be used to control expression of a coding sequence, wherein the promoter is not natively associated with the coding sequence.

Specific expression constructs described herein comprise a coding sequence linked with a leader sequence (e.g., a secretion signal peptide sequence (pre-sequence), a pro-sequence, or a pre-pro-sequence), which causes transport of the expressed polypeptide or protein into the secretory pathway and/or secretion from the host cell. The presence of such a secretion leader sequence in an expression cassette or construct is typically required when the polypeptide or protein intended for recombinant expression is not naturally secreted by the host cell and therefore lacks a natural secretion leader sequence, or its nucleotide sequence has been cloned without its natural secretion leader sequence.

In specific embodiments, multicloning vectors may be used, which are vectors having a multicloning site. Specifically, one or more desired polynucleotides can be integrated or incorporated at a multicloning site to prepare an expression vector. In the case of multicloning vectors, a promoter is typically placed upstream of the multicloning site to control expression of said one or more desired polynucleotides.

The term “gene expression”, “expressing a polynucleotide”, “expressing a nucleic acid molecule” or “expressing a pathway” as used herein, is meant to encompass at least one step selected from the group consisting of DNA transcription into mRNA, mRNA translation and processing, mRNA maturation, mRNA export, protein folding and/or protein transport, thereby obtaining expression products, precursors or intermediates thereof.

The terms “polynucleotide”, “nucleic acid molecule(s)” or “nucleic acid sequence(s)” are interchangeably used herein and refer to nucleotides, either ribonucleotides or deoxyribonucleotides or a combination of both, in a polymeric unbranched form of any length. Preferably, a polynucleotide refers to deoxyribonucleotides in a polymeric unbranched form of any length. Here, nucleotides consist of a pentose sugar (deoxyribose), a nitrogenous base (adenine, guanine, cytosine or thymine) and a phosphate group.

The term “endogenous” as used herein is meant to include those molecules and sequences, in particular genes or proteins, which are present in a wild-type (native, not recombinant) host cell or expressed by such wild-type host cell, thereby “endogenous” to said wild-type host cell. In particular, an endogenous nucleic acid molecule (e.g., a gene) or protein that does occur in (and originates from, or can be obtained from) a particular host cell as it is found in nature, is understood to be “host cell endogenous” or “endogenous to the host cell”. Moreover, a cell “endogenously expressing” a nucleic acid or protein expresses that nucleic acid or protein as does a host of the same particular type as it is found in nature. Moreover, a host cell “endogenously producing” or that “endogenously produces” a nucleic acid, protein, or other compound produces that nucleic acid, protein, or compound as does a host cell of the same particular type as it is found in nature. An endogenous protein can be overexpressed such as e.g., in an engineered host cell comprising an artificial expression cassette that expresses the protein at a higher level as compared to the level expressed by a respective wild-type host cell without such engineering. Even if a protein is no more produced by a host cell, such as e.g., a knockout mutant of the host cell, where the protein encoding gene is inactivated or deleted, the protein is herein still referred to as “endogenous”.

The term “heterologous” as used herein with respect to a nucleotide sequence, an expression cassette or construct, or any element or part of any one of the foregoing, an amino acid sequence or protein, refers to a compound which is either foreign to a given host cell, i.e. “exogenous”, such as not found in nature in said host cell; or that is naturally found in a given host cell, e.g., is “endogenous”, however, in the context of a heterologous construct or integrated in such heterologous construct, e.g., employing a heterologous nucleic acid fused or in conjunction with an endogenous nucleic acid, thereby rendering the construct heterologous. The heterologous nucleotide sequence as found endogenously may be produced in an unnatural, e.g., greater than expected or greater than naturally found, amount in the cell. The heterologous nucleotide sequence, or a nucleic acid comprising the heterologous nucleotide sequence, possibly differs in sequence from the endogenous nucleotide sequence but may still encode the same protein as found endogenously. Specifically, heterologous nucleotide sequences are those not found in the same relationship to a host cell in nature. Any recombinant or artificial nucleotide sequence is understood to be heterologous. An example of a heterologous polynucleotide is a fusion product of a nucleotide sequence encoding an FLS that is fused to a PTS coding nucleotide sequence to which the respective endogenous, naturally-occurring FLS coding sequence is not normally fused, or a nucleotide sequence operably linked to a promoter to which the respective endogenous, naturally-occurring nucleotide sequence is not normally operably linked.

The term “formolase”, abbreviated FLS, as used herein shall refer to an enzyme which catalyzes carboligation of three 1-carbon formaldehyde molecules into one 3-carbon dihydroxyacetone molecule.

An FLS may originate from a natural source, or can be a functional mutant thereof. Alternatively, an FLS can be a computationally-designed enzyme derived from benzaldehyde lyase (BAL).

The FLS may comprise or consist of any one of SEQ ID NO:31 or SEQ ID NO:32, or be a functional variant of any of the foregoing. The FLS may be provided as a derivative of any of the foregoing e.g., a fusion protein comprising the FLS sequence and a PTS.

The term “methanol to formaldehyde converting enzyme”, abbreviated “MFCE”, as used herein shall refer to an “alcohol oxidase”, abbreviated “AOX”, or “methanol oxidase”, or “methanol dehydrogenase”, and shall refer to an enzyme which catalyzes conversion of methanol into formaldehyde. Unlike alcohol dehydrogenases, alcohol oxidases are unable to catalyze the reverse reaction. Reference is made to EC 1.1.3.13 (alcohol oxidase) and EC 1.1.1.1 (alcohol dehydrogenase).

An MFCE may originate from a natural source, such as originating from a methylotrophic yeast, or can be a functional mutant thereof. Alternatively, an MFCE can be a computationally-designed enzyme derived from a functional oxidoreductase or dehydrogenase.

The MFCE may comprise or consist of any one of SEQ ID NO:27, 28, 29 or 30, or be a functional variant of any of the foregoing. A “functional mutant” or “functional variant” of an enzyme like FLS or MFCE, as used herein shall mean a variant in which at least one amino acid among wild-type enzyme amino acids is substituted, inserted, removed or modified, preferably wherein the mutation is a limited number of point mutations (a point mutation being the substitution, insertion, removal or modification of only one amino acid in a sequence), such as up to 10, 9, 8, 7, 6, 5, 4, 3, 2, or 1 point mutation(s), and/or wherein the enzyme mutant comprises a certain sequence identity to the wild-type sequence, such as at least any one of 80%, 85%, 90%, 95%, 96%, 97%, 98%, 99% or 100% sequence identity.

The term “isolated” as used herein with respect to a polynucleotide, nucleic acid molecule, expression cassettes or constructs, or host cells shall refer to such compound that has been sufficiently separated from the environment with which it would naturally be associated. Yet, “isolated” does not necessarily mean the exclusion of artificial or synthetic mixtures with other compounds or materials, or the presence of impurities that do not interfere with the fundamental activity, and that may be present, for example, due to incomplete purification. Isolated compounds can be further formulated to produce preparations thereof, and still for practical purposes be isolated. For example, an isolated expression product can be mixed with pharmaceutically acceptable carriers or excipients when used in diagnosis or therapy.

The term “metabolite” as used herein refers to a product of metabolic reactions catalyzed by one or more enzymes of a metabolic pathway of a host cell incorporating the pathway, and may include reactant, product and cofactor molecules of said enzymes. Metabolites may arise in the same pathway(s) as the cell metabolic pathway or pathways encoding an enzyme which catalyzes the synthesis of the cell growth and/or productivity inhibitor or intermediate thereof or may be synthesized in a branching pathway.

The term “operably linked” as used herein refers to the association of nucleotide sequences on a single nucleic acid molecule, e.g., a vector, an expression cassette or an expression construct, in a way such that the function of one or more nucleotide sequences is affected by at least one other nucleotide sequence present on said nucleic acid molecule. By operably linking, a nucleic acid sequence is placed into a functional relationship with another nucleic acid sequence on the same nucleic acid molecule. For example, a promoter and/or a gene switch is operably linked with a coding sequence of a recombinant gene, when it is capable of effecting the expression of that coding sequence. Specifically, such nucleic acids operably linked to each other may be directly linked i.e., immediately linked, meaning without additional elements or nucleic acid sequences in between the nucleic acids. Alternatively, a suitable linking sequence can be used such as e.g., a cloning site positioned between the promoter and/or gene switch and a gene of interest e.g., a target gene.

A promoter sequence is typically understood to be operably linked to a coding sequence, if the promoter controls the transcription of the coding sequence. If a promoter sequence is not natively associated with the coding sequence, its transcription is either not controlled by the promoter in native (wild-type) cells or the sequences are recombined with different contiguous sequences.

The term “promoter” as used herein is meant to refer to a nucleic acid molecule that initiates, regulates, or otherwise mediates or controls the expression of a protein coding polynucleotide (DNA). Promoter DNA and/or coding DNA may be native DNA such as occurring or originating from a wild-type cell e.g., from the same gene or from different genes, or may be from the same or different organisms, but can be recombinant or artificial. A heterologous promoter may be heterologous to a polynucleotide to be expressed and/or an artificial promoter, or a promoter that is originating from the wild-type host cell, but positioned in the host cell genome within a heterologous expression cassette or positioned at a location where it is not naturally-occurring in the wild-type host cell.

Specific promoters are regulatable e.g., inducible or repressible by a compound, or constitutive.

Exemplary promoters which can be used for the purpose described herein to express the recombinant FLS in the eukaryotic host cell are selected from the group consisting of methanol-inducible promoters, such as the AOX1 or AOX2 promoter, or a constitutive promoter such as MDH3, POR1, PDC1, FBA1-1, or GPM1 (Prielhofer et al. 2017, BMC Sys Biol. 11: 123), or a functional variant of any of the foregoing.

As an alternative to native or wild-type promoter sequences, functional variants of such native or wild-type promoter sequences (herein understood as parent promoters) can be used, which have at least any one of 80%, 85%, 90%, 95%, 96%, 97%, 98%, 99% or 100% sequence identity and are functional in controlling the expression of a gene in substantially similar way, e.g. being an inducible promoter or constitutive promoter as the parent promoter, and preferably having about the same or increased promoter strength.

Specifically, a functional variant of a promoter described herein comprises a certain sequence identity to the promoter from which it is derived, over the full-length or the part at the 3′-end of the promoter sequence which part has a length of at least 300, 400, or 500 bp, and which is functional to operatively control expression of the polynucleotide to be expressed, in particular with about the same promoter activity (e.g. +/−any one of 50%, 40%, 30%, 20%, or 10%), although the promoter activity may be increased to higher than 100% as compared to the promoter from which it is derived.

The promoter activity can be determined by measuring the strength of the promoter. The promoter strength is typically understood as the transcription strength. There are standard methods to determine the transcription strength of a promoter, such as by measuring the quantity of transcripts, e.g., employing a microarray, or else in a cell culture, such as by measuring the quantity of respective gene expression products in recombinant cells. In particular, the transcription rate may be determined by the transcription strength on a microarray, Northern blot or with quantitative real time PCR (qRT-PCR) or with RNA sequencing (RNA-seq).

A “methanol-inducible promoter” is herein understood as a naturally occurring or wild-type promoter controlling expression of genes of the methanol dissimilatory pathway of organisms, in particular methylotrophic microorganisms.

For the purpose described herein, expression of the recombinant FLS may be driven by a methanol-inducible promoter, such as the AOX1 or AOX2 promoter, or a functional variant of any of the foregoing.

According to the methanol dissimilatory pathway in methylotrophic yeast, such as P. pastoris, methanol passively diffuses into the yeast peroxisome. There it is converted to formaldehyde by one of two different alcohol oxidase isozymes (Aox1, Aox2). In a wild-type methylotrophic yeast, formaldehyde can be further oxidized in several steps to CO₂ via the methanol dissimilatory pathway. Alternatively, formaldehyde is incorporated into the pentose phosphate pathway via a condensation reaction with xylulose 5-phosphate, a reaction catalyzed by a specialized transketolase enzyme called DiHydroxyAcetone Synthase (Das). This reaction yields a molecule of dihydroxyacetone (DHA) and a molecule of glyceraldehyde 3-phosphate. Each of these reactions occurs in peroxisomes in methylotrophic yeasts. As an alternative to such wild-type pathways, formaldehyde is directly converted to DHA by the recombinant FLS in an engineered cell as described herein.

The term “nucleotide sequence” or “nucleic acid sequence” as used herein refers to either DNA or RNA. “Nucleic acid sequence” or “polynucleotide sequence” or simply “polynucleotide” refers to a single or double-stranded polymer of deoxyribonucleotide or ribonucleotide bases read from the 5′ to the 3′ end. It includes expression cassettes, self-replicating plasmids, infectious polymers of DNA or RNA, and non-functional DNA or RNA.

The term “peroxisomal targeting signal” (PTS) as used herein shall refer to short nucleic acid sequences which when linked to or positioned within a coding sequence, e.g. as a nucleotide sequence encoding a C-terminal peptide or an N-terminal peptide of typically 3-9 amino acids, directs the expression of the expression product to the peroxisome of the host cell. By such a functional PTS, an enzyme can be relocated to the peroxisome. Most organisms including Pichia pastoris have two different targeting systems. The first one (PTS1) uses the receptor Pex5 to achieve targeting to the peroxisome. The second one (PTS2) uses Pex7 as receptor. A functional PTS is an amino acid sequence which is specifically recognized by any of the receptors Pex5 (PTS1) or Pex7 (PTS2), thereby activating the receptor and directing expression of the gene that is fused with such PTS to the host cell peroxisome.

A nucleotide sequence encoding the PTS1 is typically linked to a gene at the 3′-end, such that the PTS is fused at the carboxy terminus of the respective gene expression product. Thereby, the C-terminus of the amino acid sequence of the gene expression product is directly linked to the N-terminus of the PTS.

A nucleotide sequence encoding the PTS2 is typically linked to a gene at the 5′-end or integrated in proximity to the 5′-end, such that the PTS is fused at the amino terminus or close to the amino terminus of the respective gene expression product. Thereby, the N-terminus of the amino acid sequence of the gene expression product is directly linked to the C-terminus of the PTS2.

The following tools can be used to determine targeting signals in a given protein sequence: PTS1 predictor (Neuberger G, Maurer-Stroh S, Eisenhaber B, Hartig A, Eisenhaber F. Motif refinement of the peroxisomal targeting signal 1 and evaluation of taxon-specific differences. J Mol Biol. 2003 May 2; 328(3):567-79.), or PTS prediction tool WoLF PSORT (Horton P, Park K-J, Obayashi T et al. WoLF PSORT: protein localization predictor. Nucleic Acids Res 2007; 35:W585-7.).

The term “protein of interest” or “(POI)” as used herein refers to a polypeptide or a protein that is produced by means of recombinant technology in a host cell. More specifically, the protein may either be a polypeptide not naturally-occurring in the host cell, i.e. a heterologous protein, or else may be native to the host cell, i.e. a endogenous protein to the host cell, but is produced, for example, by transformation or transfection with a self-replicating vector containing the nucleic acid sequence encoding the POI, or upon integration by recombinant techniques of one or more copies of the nucleic acid sequence encoding the POI into the genome of the host cell, or by recombinant modification of one or more regulatory sequences controlling the expression of the gene encoding the POI, e.g., of the promoter sequence.

The term “sequence identity” of a variant, homologue or orthologue as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or genes by detecting excess similarity, and statistically significant similarity that reflects common ancestry. Homologues may encompass orthologues, which are herein understood as the same protein in different organisms, e.g., variants of such protein in different different organisms or species.

“Percent (%) amino acid sequence identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

For purposes described herein, the sequence identity between two amino acid sequences can be determined using the NCBI BLAST program version BLASTP 2.8.1 with the following exemplary parameters: Program: blastp, Word size: 6, Expect value: 10, Hitlist size: 100, Gapcosts: 11.1, Matrix: BLOSUM62, Filter string: F, Compositional adjustment: Conditional compositional score matrix adjustment.

For pairwise protein sequence alignment of two amino acid sequences along their entire length the EMBOSS Needle webserver can be used with default settings (Matrix: EBLOSUM62; Gap open:10; Gap extend: 0.5; End Gap Penalty: false; End Gap Open: 10; End Gap Extend: 0.5). EMBOSS Needle uses the Needleman-Wunsch alignment algorithm to find the optimum alignment (including gaps) of the two input sequences and writes their optimal global sequence alignment to file.

“Percent (%) identity” with respect to a nucleotide sequence e.g., of a coding or non-coding nucleotide sequence, is defined as the percentage of nucleotides in a candidate nucleotide sequence that is identical with the nucleotides in the nucleotide sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

For purposes described herein (unless indicated otherwise), the sequence identity between two amino acid sequences can be determined using the NCBI BLAST program version BLASTN 2.8.1 with the following exemplary parameters: Program: blastn, Word size: 11, Expect threshold: 10, Hitlist size: 100, Gap Costs: 5.2, Match/Mismatch Scores: 2, -3, Filter string: Low complexity regions, Mark for lookup table only.

The term “recombinant” as used herein shall mean “being prepared by or the result of genetic engineering. A “recombinant cell” or “recombinant host cell” is herein understood as a cell or host cell that has been genetically engineered or modified to comprise a nucleic acid sequence which was not native to said cell. A recombinant host may be engineered to delete and/or inactivate one or more nucleotides or nucleotide sequences, and may specifically comprise an expression vector or cloning vector containing a recombinant nucleic acid sequence, in particular employing nucleotide sequence foreign to the host. A recombinant protein is produced by expressing a respective recombinant nucleic acid in a host. The term “recombinant” as used herein with respect to expression products, includes those compounds that are prepared, expressed, created or isolated by recombinant means, such as isolated from a host cell transformed or transfected to express the expression products.

Certain recombinant host cells are “engineered” host cells which are understood as host cells which have been manipulated using genetic engineering, i.e. by human intervention. When a host cell is engineered to express, co-express or overexpress a certain pathway, gene or the respective protein, the host cell is manipulated such that the host cell has the capability to express such pathway, gene and protein, respectively, to a different extent compared to the host cell under the same condition prior to manipulation, or compared to the host cells which are not engineered such that said gene or protein is expressed, co-expressed or overexpressed.

The term “synthetic” as used herein in the context with a synthetic pathway shall mean a pathway that is recombinant.

As herein described, it was found that the methanol assimilation pathway (Xylulose-Monophosphate cycle, XuMP) is compartmentalized in peroxisomes and has similarities to the Calvin-Benson-Bassham cycle and the pentose-phosphate pathway (RuBmayer et al., 2015). However, this pathway requires at least 10 enzymes to enable the conversion from methanol to DHA. In addition, three (3) moles of ATP are required for the conversion of three moles of (3) formaldehyde to one (1) mol of Dihydroxyacetone phosphate (DHAP). In order to produce ATP a significant amount of methanol has to be dissimilated by a cell such as P. pastoris to CO₂ obtaining NADH and thus ATP via the respiratory chain. The formation of dihydroxyacetone phosphate is thus not as energy efficient as the new FLS pathway described herein which can directly form dihydroxyacetone from three (3) moles of formaldehyde. Then only one (1) mol of ATP is required (instead of three mol ATP) to form dihydroxyacetone phosphate, which is an intermediate of glycolysis and thus of the central carbon metabolism. Therefore, a recombinant pathway was implemented into eukaryotic cells, in particular microorganisms, to enable a formaldehyde conversion into biomass via the formose reaction. This can enable an energy efficient conversion of C₁ building blocks into carbon molecules with multiple C—C bonds.

According to a specific example, an FLS gene (version FLS-M3) as published by (Cai et al. 2021) was codon optimized for Komagataella phaffii and a PTS1 sequence was added to the C-terminus to enable targeting to peroxisomes of K. phaffii. The coding sequence was placed under the control of a strong methanol inducible promoter (either pAOX1 or pDAS2) and expression cassettes were integrated into a K. phaffii strain which has a deletion in DAS1 and DAS2 (Mut- strain). A Mut- strain is not capable to grow on methanol as carbon source. The presence of the expression cassette can be checked by for instance PCR approaches. Obtained strains can grow on medium with methanol as single carbon source and show formolase activity in a cell-free extract.

The formolase pathway described herein enables a more efficient utilization of a C₁ (one carbon) carbon source like methanol. Methanol is an important carbon source to grow methylotrophic yeasts like Pichia pastoris (Komagataella sp.) to produce mainly proteins and enzymes. Furthermore, the production of single cell proteins for food and feed applications is in the industrial focus again. With a more efficient methanol assimilation a higher yield of the product formed can be achieved (e.g., proteins, organic acids, DNA, RNA, lipids, biomass, . . . ). Furthermore, as the pathway is more efficient less heat development is expected to occur when this pathway is operated. Cooling bioreactors to e.g., 30 °C is cost intensive when an exothermic reaction is taking place.

Formaldehyde is a reactive molecule, which is in many cases cytotoxic to microorganism when formed intracellularly at higher concentrations or without specialized compartmentalization in the cell. According to a specific example, to establish a formolase pathway in vivo, an intracellular compartment of the cell is utilized (or generated), where both formaldehyde is formed and converted by formolase to dihydroxyacetone.

According to a specific aspect, biochemical reactions leading to the formation of formaldehyde are placed into the same compartment as the formose reaction carried out by the enzyme formolase (FLS) to form dihydroxyacetone from formaldehyde. Hereby, a compartment is a subcellular container, which is spatially separating its interior from the surrounding environment by a biochemical barrier. This biochemical barrier can be a lipid membrane consisting of phospholipids (e.g., organelles like peroxisomes, mitochondria, vacuole, endoplasmic reticulum) or an artificial shell formed by protein structures like in bacterial microcompartments.

The FLS pathway as described herein is using formaldehyde as a substrate or intermediate. Preferably, the FLS pathway is spatially localized to the same subcellular compartment like the peroxisome. Doing so other cellular compounds like DNA, RNA or proteins are not modified by formaldehyde in an untargeted way.

The foregoing description will be more fully understood with reference to the following examples. Such examples are, however, merely representative of methods of practicing one or more embodiments of the present invention and should not be read as limiting the scope of invention.

### EXAMPLES

### Example 1: Construction of FLS Coding Sequence with a Peroxisomal Targeting Sequence

An improved version of formolase (FLS), which was described as FLS-M3 (Cai et al., 2021) was targeted to the peroxisome of Pichia pastoris by fusing a peroxisomal targeting sequence to the C-terminus of the protein. The targeting sequence, derived from the C-terminus of the native AOX1 of P. pastoris (represented in the protein dodecamer: LGTYEKTGLARF, SEQ ID NO:19, termed PTS1 here) was fused to the C-terminus of FLS (protein sequence: SEQ ID NO:2) yielding FLS::PTS1. The respective protein sequence was codon optimized for P. pastoris and the resulting DNA sequence was ordered at TWIST Bioscience (South San Francisco, CA, USA) (DNA sequence: SEQ ID NO:1). Alternatively other targeting signals for peroxisomal localization can be added to the protein, which targets the PEX5 receptor (mainly PTS1) or like PTS2 (via PEX5 or PEX7 receptor) or PTS3 (Kempifski et al., 2020). Proteins showing a peroxisomal localization were reported previously together with respective targeting signals (Rußmayer et al., 2015).

The coding sequence was ordered with flanking Bpil (Bbsl) sites and respective fusion sites (FS2, FS3) to be compatible with a Golden Gate cloning pipeline GoldenMOCS described previously (Sarkari et al., 2017).

### Example 2: Construction of DNA Cassettes and Generation of FLS Expressing Yeast Strains in the Peroxisome

The genotype of the parental strain (Komagataella phaffii CBS7435 ΔDAS1, ΔDAS2, ΔAOX1=MUT⁻) was determined by amplifying the CDS of DAS1, DAS2, and AOX1 from isolated genomic DNA in a PCR reaction using 05 High-Fidelity DNA Polymerase (NEB) using SEQ ID NO:5, SEQ ID NO:6, SEQ ID NO:7, SEQ ID NO:8, SEQ ID NO:9, SEQ ID NO:10 as the primer oligonucleotide pairs. The sequencing results (SEQ ID NO:13, SEQ ID NO:14, and SEQ ID NO:15) confirmed the deletion of the mentioned genes in the genome of the parental Pichia pastoris MUT⁻ strain. This deletion strain cannot grow on methanol. The methanol assimilation pathway (Xylulose-Monophosphate cycle) is blocked when both DAS1/DAS2 are deleted (Gassler et al., 2020).

In this strain background the fusion protein FLS::PTS1 was expressed using a strong inducible methanol promoter using pDAS2 or pAOX1 by integrating respective expression cassettes into the genome.

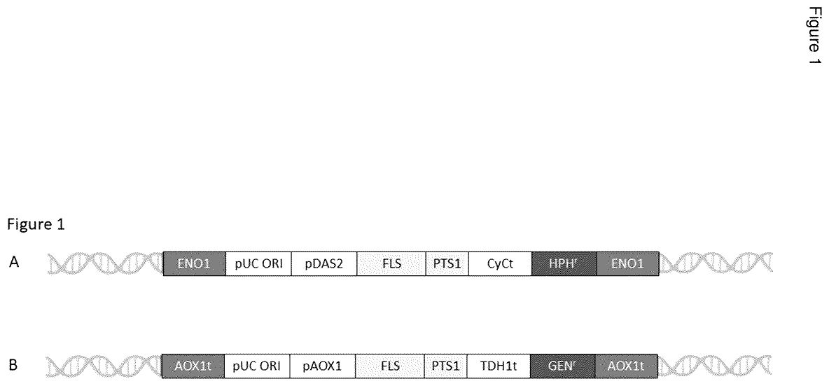

The integration cassettes (FIG. 1) were constructed using a Golden gate cloning strategy GoldenMOCS and transformed to electro-competent P. pastoris ΔDAS1, ΔDAS2, ΔAOX1 cells. 5 μg DNA was transformed to each competent cell. The final incubated cells were plated on YPD-Agar plates having 500 μg mL⁻¹ G418 or 200 μg mL⁻¹ Hygromycin as antibiotic selection markers.

After 48 hours of incubation at 28 °C the single colonies were picked from plates and were streaked out twice on antibiotic plates (Hygromycin, G418) and once on M2 Media+0.5% Methanol+1.5% Agar.

The transformants that grew on both antibiotic (Hygromycin or G418) and methanol plates were selected. A cell bank of the selected strain was prepared, in 1 mL aliquots and stored at −80 °C. The extraction of the genomic DNA in order to check the integration of the genes of interest in the genome was done by breaking the cells down using glass beads in a Bead ruptor device (FastPrep-24, MP Biomedical, 6 m/s) and genomic DNA was isolated using an ethanol precipitation protocol (Promega kit).

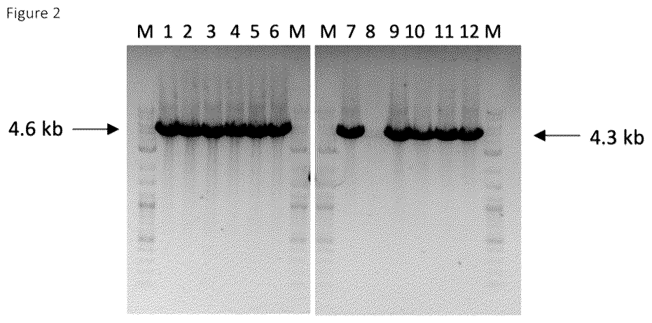

The SEQ ID NO:3 and SEQ ID NO:4 were amplified from yeast genomic DNA in PCR reactions using OneTaq DNA polymerase (NEB) with SEQ ID NO:11 and SEQ ID NO:12 as the primer oligonucleotides. The DNA fragments sized 4.6 kb for SEQ ID NO:3 (pDAS2::FLS::HpHr) and 4.3 kb for SEQ ID NO:4 (pAOX1::FLS::G418r) were expected. The presence of the mentioned fragments was checked on 1% Agarose gel (FIG. 2). The positive P. pastoris transformants having SEQ ID NO:4 (FIG. 2, Lane 1-6) or SEQ ID NO:5 (FIG. 2, Lane 7,9-12) integrated into their genomes were selected and used for further experiments.

### Example 3: Cultivation of FLS Expressing Yeast Strains on Synthetic Media Containing Methanol

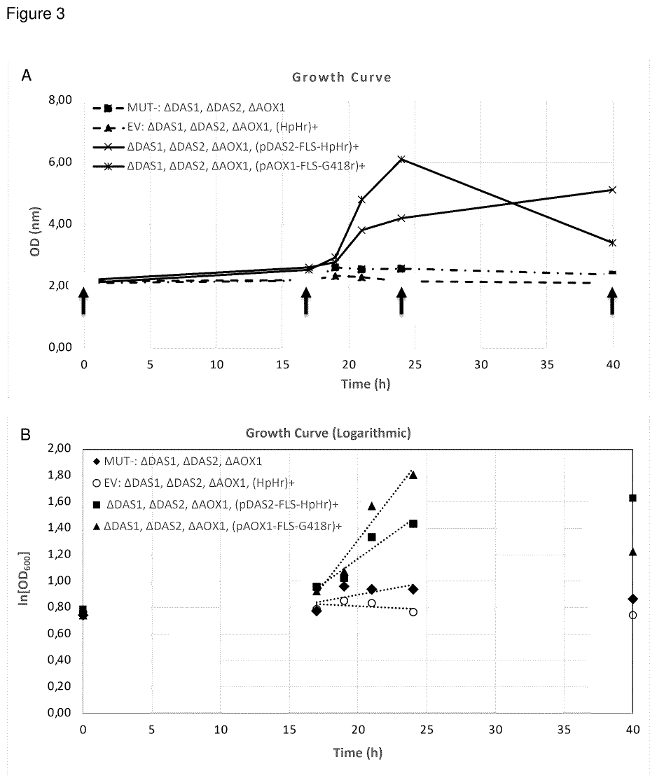

One single colony from each of the described P. pastoris transformants from YPD agar plates was taken and inoculated in shake flasks containing 40 mL of YPD medium, cultivated overnight at 28 °C in a shaking incubator at 200 rpm. The next day the yeast cells were collected at 5000 g, for 5 min and resuspended in 20 mL M2 Media without a carbon source. The optical density (OD) was measured at 600 nm and the cells were inoculated to the 50 mL M2 media (in wide-neck 500 mL flasks with cotton-wools) containing 5 g L⁻¹ methanol to reach the starting OD of 2.5. The flasks were incubated at 28 °C at 200 rpm. The growth of the yeast cultures on methanol as the sole carbon source were monitored for the period of 40 hours by measuring OD at the time points shown in FIG. 3. The methanol feeding strategy is shown in Table 1. The obtained growth rate is shown in Table 2.

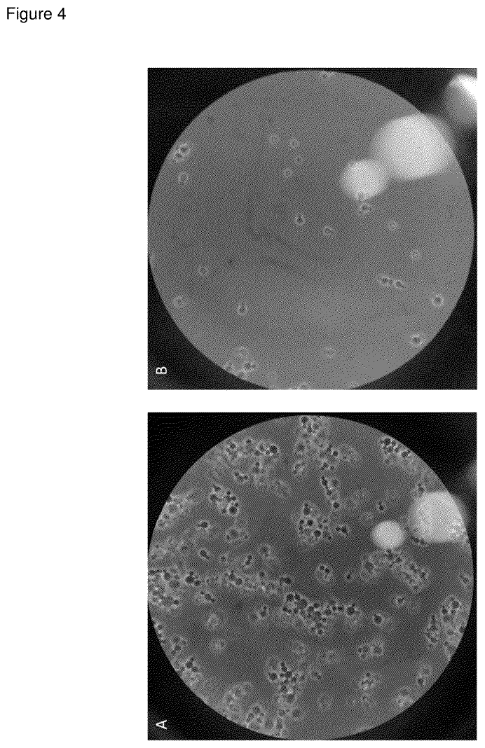

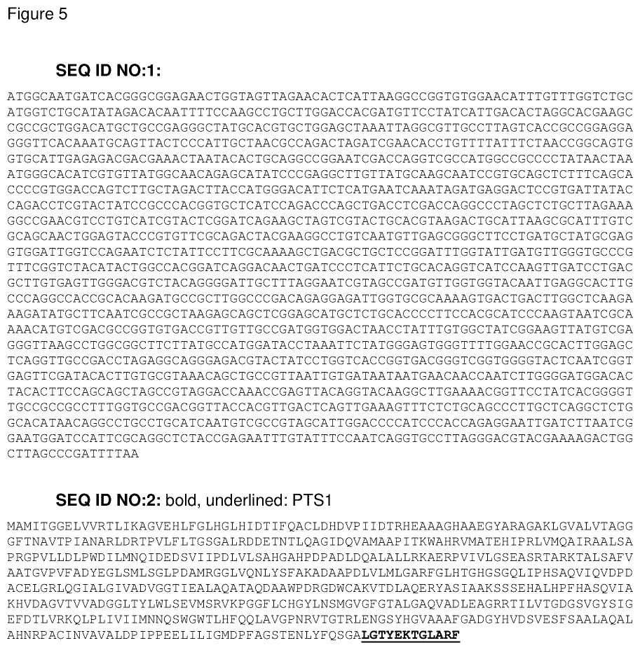

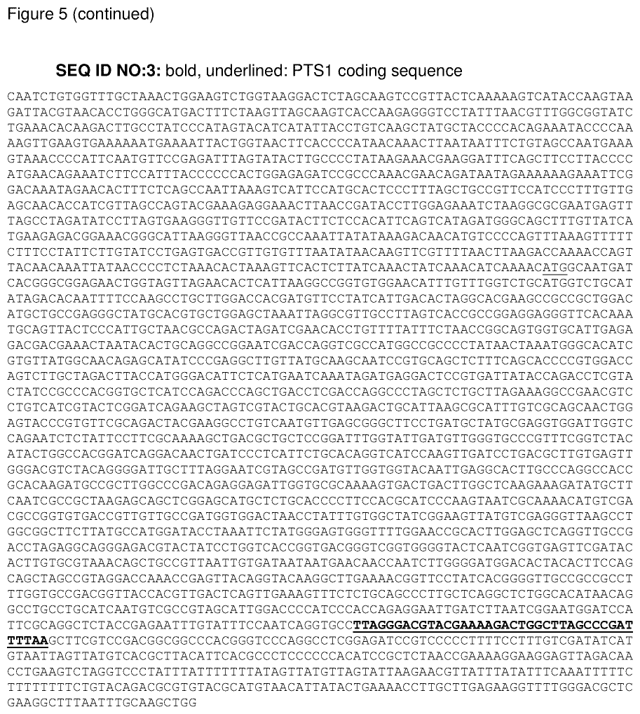

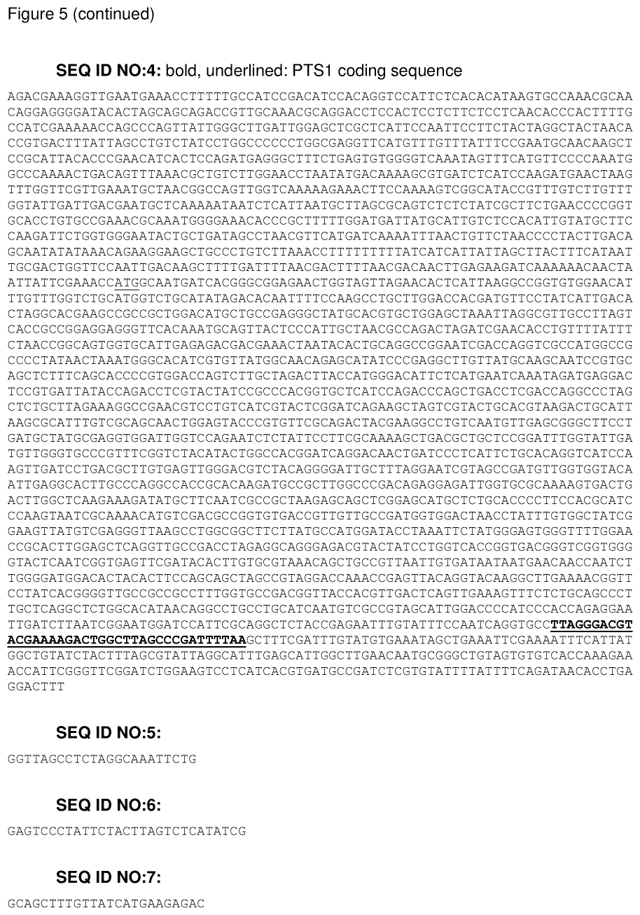

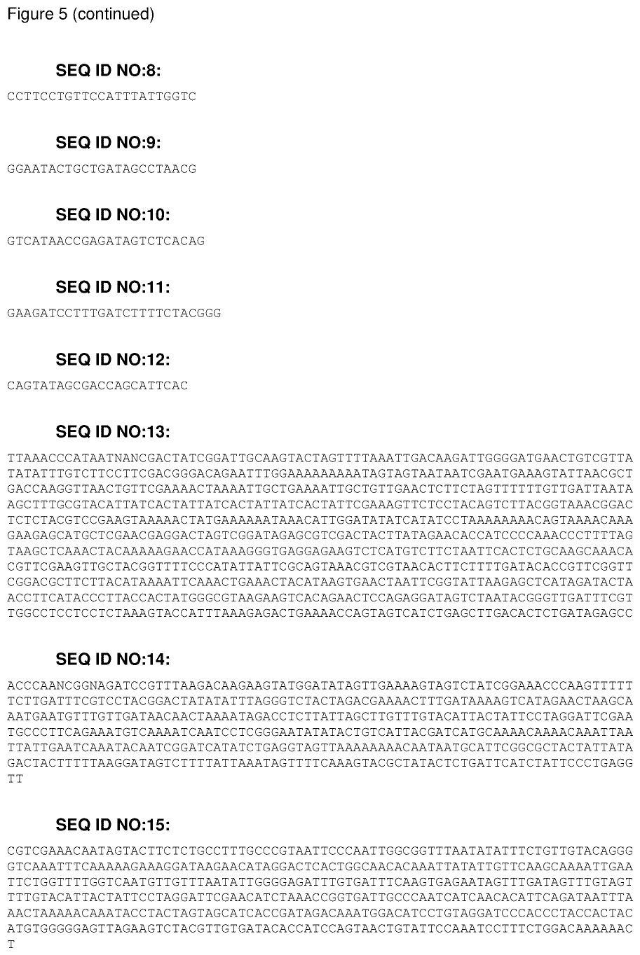

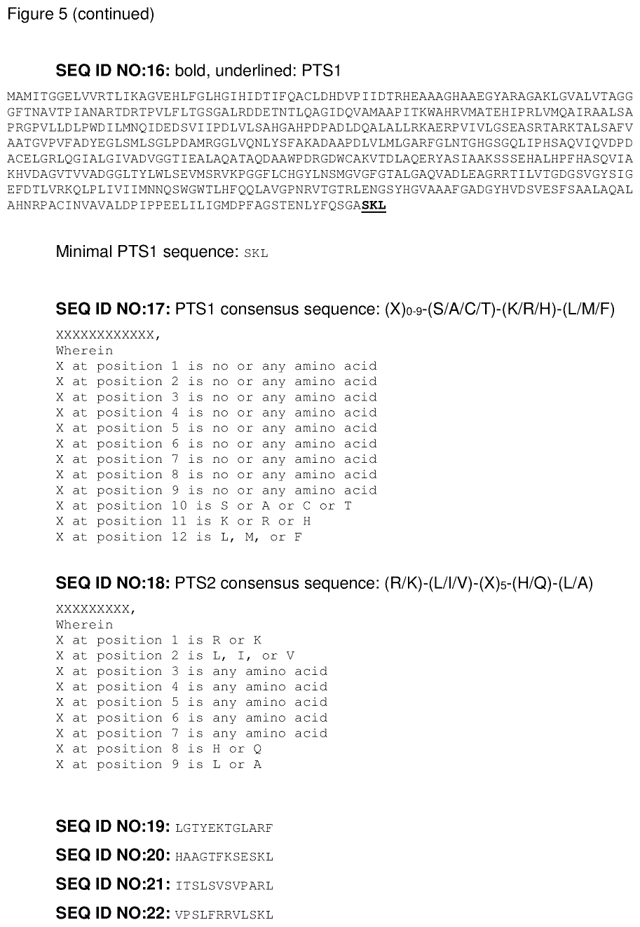

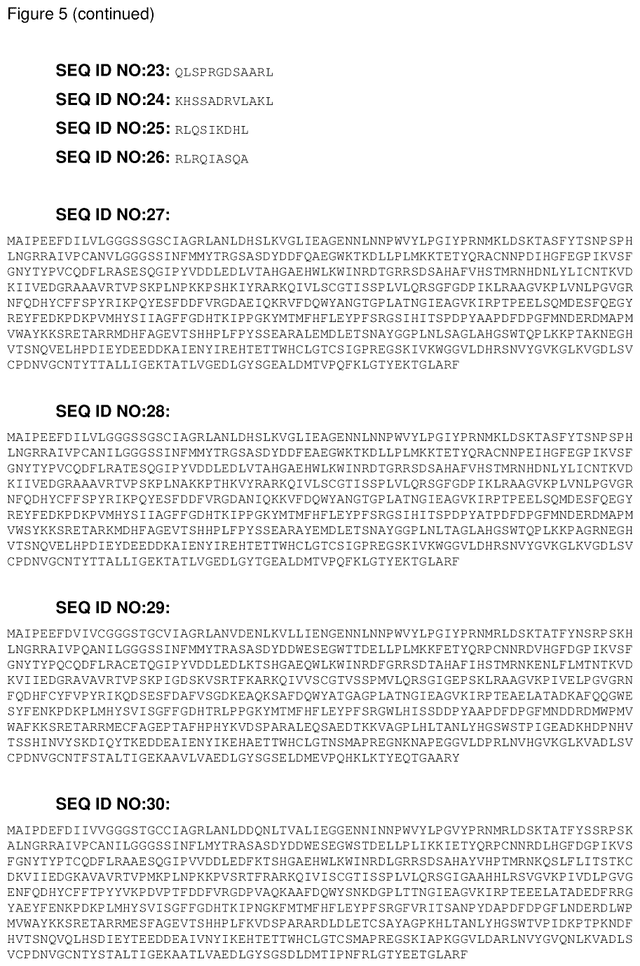

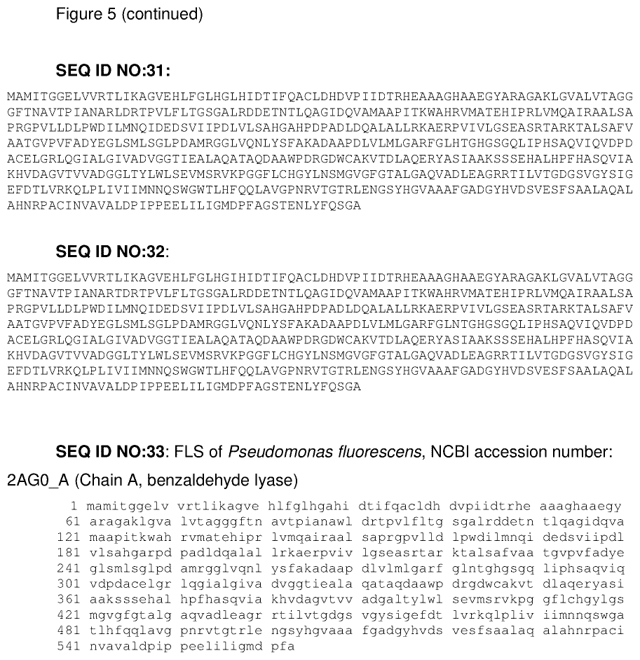

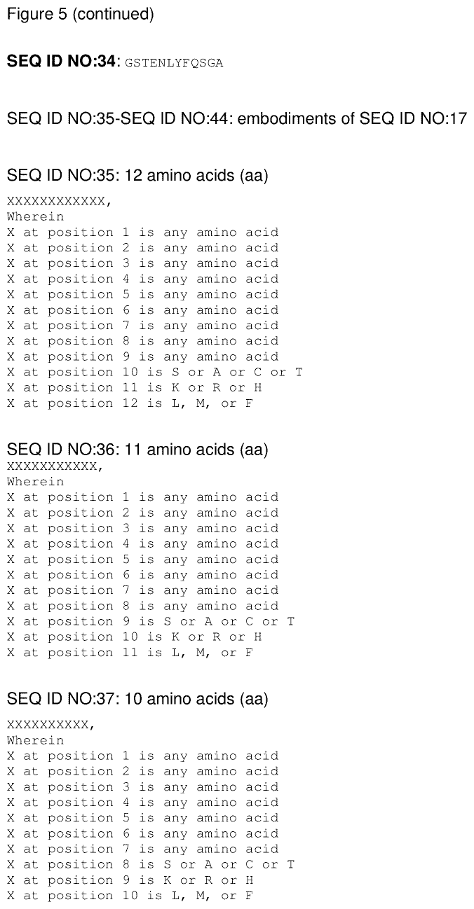

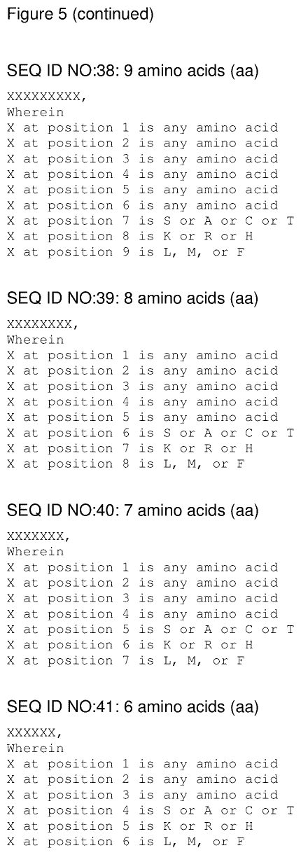

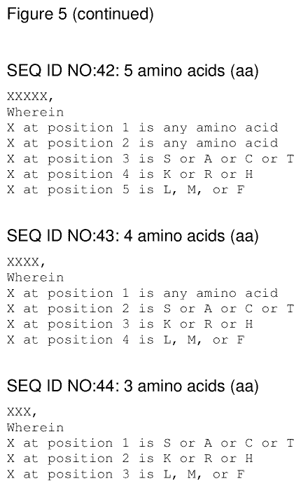

Strains expressing FLS can grow on methanol as sole carbon source. No growth is obtained for an empty vector control and the parental MUT⁻ strain on methanol. From FIG. 4 it can be seen that strains expressing FLS show a dense and budding yeast culture on methanol under the microscope compared to the not growing parental strain.

### Media Compositions:

YPD Media: 1% yeast extract, 2% peptone, 2% glucose.

Synthetic Media (M2 Citrate buffered): Per liter; 3.15 g (NH₄)₂HPO₄, 0.49 g MgSO₄·7H₂0, 0.80 g KCl, 0.0268 g CaCl₂·2H₂O, 22.0 g citric acid monohydrate, 1.47 mL Trace salt (PTMO), 4 mL biotin (0.1 g L⁻¹), 4 mL thiamine-hydrochloride (5 g L⁻¹). The pH of the media was adjusted at 6 by adding potassium hydroxide pellets.

Trace salt solution (PTMO): Per liter; 5 mL sulphuric acid (95%-98%), 65 g FeSO₄·7H₂O, 20 g ZnCl₂, 6 g CuSO₄·5H₂O, 3.36 g MnSO₄·H₂O, 0.82 g CoCl₂·6H₂O, 0.2 g Na₂MoO₄·2H₂O, 0.08 g NaI, 0.02 g H₃BO₃.

TABLE 1
Methanol feeding strategy in 40 hours cultivation of
P. pastoris strains in synthetic M2/methanol media

| Time (h) | MeOH feeding |
|---|---|
| 0 | 0.50% |
| 17 | 0.60% |
| 19 | 0 |
| 21 | 0 |
| 24 | 0.50% |
| 40 | 0.60% |

TABLE 2
Comparison of the growth and enzyme activity of
P. pastoris MUT- parental strain compared to
P. pastoris FLS expressing transformants

| | Growth | FLS |
|---|---|---|
| | rate | enzyme |
| Genotype | (h⁻¹) | activity |
| ΔDAS1, ΔDAS2, ΔAOχ1 | 0.019 | − |
| ΔDAS1, ΔDAS2, ΔAOX1, (HpH^{r})⁺ | −0.004 | − |
| ΔDAS1, ΔDAS2, ΔAOχ1, (pDAS2-FLS-HpH^{r})⁺ | 0.074 | + |
| ΔDAS1, ΔDAS2, ΔAOX1, (pAOX1-FLS-G418^{r})⁺ | 0.13 | + |

### Example 4: Enzymatic Assay to Measure FLS Activity in Cell Free Extracts

The enzymatic assay was done by a colorimetric assay protocol (Cai et al., 2021). After cultivation in synthetic media, the yeast cell pellets were collected and resuspended in (HEPES-NaCl buffer, 100 mM pH 7.5). The cells were broken down by adding glass beads in a bead ruptor device (4 m/s). The cell free extract containing FLS enzyme was separated from the cell debris. The enzyme activity assay was carried out in microtiter plates. 50 mL of formaldehyde solution (ranging from 20 to 100 mM) containing 1 mM thiamine pyrophosphate (TPP) was added to 50 μL of cell free extract dilutions. After incubation at 30 °C, 60 μL of enzyme solution (0.3 mg mL⁻¹ galactose oxidase, 35 U mL⁻¹ horseradish peroxidase) and 50 μL of 8 mM ABTS solution was added to each well. The plate was subjected to UV/Vis measurement at 410 nm using a microtiter plate reader. The blank had the same above-mentioned reaction solutions but 50 μL buffer was added instead of cell free extract. The cell free extract from the parental strain (ΔDAS1, ΔDAS2, ΔAOX1) was measured as a negative control.

Strains expressing FLS show enzymatic activity for FLS when grown on methanol. No FLS activity is obtained for an empty vector control and the parental MUT⁻ strain.

### References (Non-Patent Literature)

Cai, T., Sun, H., Qiao, J., Zhu, L., Zhang, F., Zhang, J., Tang, Z., Wei, X., Yang, J., Yuan, Q., Wang, W., Yang, X., Chu, H., Wang, Q., You, C., Ma, H., Sun, Y., Li, Y., Li, C., Jiang, H., Wang, Q., Ma, Y., 2021. Cell-free chemoenzymatic starch synthesis from carbon dioxide. Science, Vol. 373, 1523-1527.
Gassler, T., Sauer, M., Gasser, B., Egermeier, M., Troyer, C., Causon, T., Hann, S., Mattanovich, D., Steiger, M. G., 2020. The industrial yeast Pichia pastoris is converted from a heterotroph into an autotroph capable of growth on CO₂. Nature Biotechnology, Vol. 38(2), 210-216.
Kempiński, B., Chetstowska, A., Poznański, J., Król, K., Rymer, L., Frydzińska, Z., Girzalsky, W., Skoneczna, A., Erdmann, R., Skoneczny, M., 2020. The Peroxisomal Targeting Signal 3 (PTS3) of the Budding Yeast Acyl-CoA Oxidase Is a Signal Patch. Frontiers in Cell and Developmental Biology, Vol. 8(198), 1-8.
Rußmayer, H., Buchetics, M., Gruber, C., Valli, M., Grillitsch, K., Modarres, G., Guerrasio, R., Klavins, K., Neubauer, S., Drexler, H., Steiger, M., Troyer, C., A I Chalabi, A., Krebiehl, G., Sonntag, D., Zellnig, G., Daum, G., Graf, A. B., Altmann, F., Koellensperger, G., Hann, S., Sauer, M., Mattanovich, D., Gasser, B., 2015. Systems-level organization of yeast methylotrophic lifestyle. BMC Biology, Vol. 13(80), 1-25.
Sarkari, P., Marx, H., Blumhoff, M. L., Mattanovich, D., Sauer, M., Steiger, M. G., 2017. An efficient tool for metabolic pathway construction and gene integration for Aspergillus niger. Bioresource Technology, Vol. 245, 1327-1333.
Siegel, J. B., Smith, A. L., Poust, S., Wargacki, A. J., Bar-Even, A., Louw, C., Shen, B. W., Eiben, C. B., Tran, H. M., Noor, E., Gallaher, J. L., Bale, J., Yoshikuni, Y., Gelb, M. H., Keasling, J. D., Stoddard, B. L., Lidstrom, M. E., Baker, D., 2015. Computational protein design enables a novel one-carbon assimilation pathway. Proceedings of the National Academy of Sciences U.S.A., Vol. 112(12), 3704-3709.
Van der Klei, I. J., Yurimoto, H., Sakai, Y., Veenhuis, M. 2006. Biochimica et Biophysica Acta, Vol. 1763(12), 1453-1462.
Wang, X., Wang, Y., Liu, J., Li, Q., Zhang, Z., Zheng, P., Lu, F., Sun, J., 2017. Biological conversion of methanol by evolved Escherichia coli carrying a linear methanol assimilation pathway. Bioresources and Bioprocessing, Vol. 4(41).

1. A eukaryotic cell which is engineered to express a synthetic formolase (FLS) pathway comprising a recombinant FLS to biotransform formaldehyde into dihydroxyacetone.

2. The cell of claim 1, wherein the FLS pathway further comprises one or more enzymes to biotransform a C₁ compound into formaldehyde, preferably wherein the C₁ compound is any one of methanol, formate, methane, carbon monoxide, or carbon dioxide.

3. The cell of claim 1, wherein the FLS pathway further comprises a methanol to formaldehyde converting enzyme (“MFCE”), preferably an alcohol oxidase or a methanol dehydrogenase, to biotransform methanol into formaldehyde.

4. The cell of claim 1, which is:
a) a yeast cell, preferably of a genus selected from the group consisting of Pichia, Komagataella, Hansenula, Saccharomyces, Kluyveromyces, Candida, Ogataea, Yarrowia, and Geotrichum, preferably Pichia pastoris, Komagataella phaffii, Komagataella pastoris, Komagataella pseudopastoris, Saccharomyces cerevisiae, Ogataea minuta, Kluyveromces lactis, Kluyveromyes marxianus, Yarrowia lipolytica or Hansenula polymorpha, preferably a methylotrophic yeast;
b) a cell of filamentous fungi, preferably of a genus selected from the group consisting of Aspergillus, Penicillium, Trichoderma, Neurospora, Rhizopus preferably Aspergillus niger, Aspergillus awamori Aspergillus terreus, Aspergillus oryzae, Aspergillus nidulans, Neurospora crassa, Rhizopus oryzae, Penicillium chrysogenum or Trichoderma reesei;
c) a non-human primate, human, rodent, or bovine cell, preferably a mouse myeloma (NSO)-cell line, a Chinese hamster ovary (CHO)-cell line, HT1080, H9, HepG2, MCF7, MDBK Jurkat, MDCK, NIH3T3, PC12, BHK (baby hamster kidney cell), VERO, SP2/0, YB2/0, YO, C127, L cell, COS, QC1-3, HEK-293, PER.C6, HeLA, EBI, EB2, EB3, oncolytic or hybridoma-cell line;
d) an insect cell, preferably a Sf9, Mimic™ Sf9, Sf21, High Five (BT1-TN-5B1-4), or BT1-Ea88 cell;
e) an algae cell, preferably of the genus Amphora, Bacillariophyceae, Dunaliella, Chlorella, Chlamydomonas, Cyanophyta (cyanobacteria), Nannochloropsis, Spirulina, or Ochromonas); or
f) a plant cell, preferably a cell from monocotyledonous plants, preferably maize, rice, wheat, or Setaria, or from a dicotyledonous plant, preferably cassava, potato, soybean, tomato, tobacco, alfalfa, Physcomitrella patens or Arabidopsis.

5. The cell of claim 1, wherein the FLS pathway is comprised in an organelle of the cell, preferably a peroxisome or mitochondria.

6. The cell of claim 1, wherein the FLS is expressed as an FLS fusion protein that comprises the FLS fused to a peroxisomal targeting signal (PTS), preferably wherein the PTS comprises or consists of an amino acid sequence selected from the group consisting of SKL, SEQ ID NO:17, 18, 19, 20, 21, 22, 23, 24, 25, and 26.

7. The cell of claim 1, wherein the FLS comprises of consists of SEQ ID NO:31 or SEQ ID NO:32, or a functional variant of any of the foregoing, preferably wherein
a) the functional variant of SEQ ID NO:31 comprises at least 1, 2, 3, 4, 5, 6, or 7 (all) of the point mutations A28L, W89R, R188H, N283H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:31;
b) the functional variant of SEQ ID NO:32 comprises at least 1, 2, 3, 4, 5, 6, or 7 (all) of the point mutations A281, W89R, L90T, R188H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:32.

8. The cell of claim 1, which is an engineered Mut- yeast, preferably engineered to reduce expression of a dihydroxyacetone synthase (DAS) compared to an endogenous expression thereof, preferably by a deletion of any one or both of the DAS1 and DAS2 genes.

9. The cell of claim 1, which is capable of utilizing a C₁ compound as the sole carbon source for producing biomass and/or an expression product.

10. A method of culturing the cell of claim 1 in a cell culture using a C₁ compound as a carbon source, thereby obtaining biomass and/or an expression product in the cell culture, preferably wherein the C₁ compound is the sole carbon source.

11. The method of claim 10, wherein the expression product is a protein of interest (POI) or a metabolite, preferably wherein:
a) the POI is a peptide or protein selected from the group consisting of an antigen-binding protein, a therapeutic protein, an enzyme, a peptide, a protein antibiotic, a toxin fusion protein, a carbohydrate-protein conjugate, a structural protein, a regulatory protein, a vaccine antigen, a growth factor, a hormone, a cytokine, a process enzyme;
b) the metabolite is an amino acid, organic acid, alcohol, sugar alcohol, carbohydrate, vitamin, amine, aldehyde, ketone, or a polyhydroxyketone, preferably wherein the metabolite is selected from the group consisting of lactic acid, citric acid, propionic acid, butyric acid, valeric acid, hexanoic acid, adipic acid, succinic acid, fumaric acid, malic acid, 2,5-furan dicarboxylic acid, aspartic acid, glucaric acid, gluconic acid, glutamic acid, itaconic acid, levulinic acid, acrylic acid, 3-hydroxy propionic acid, ethanol, propanol, isopropanol, butanol, pentanol, hexanol, heptanol, octanol, butanediol, 1,3-propanediol, 1,2-propanediol, 2-amino-1,3-propanediol, 3-hydroxybutyrate, poly-3-hydroxybutyrate, 3-hydroxy propionaldehyde, 3-hydroxybutyrolactone, xylitol, arabinitol, sorbitol, mannitol, vitamin C, riboflavin, thiamine, tocopherol, cobalamin, pantothenic acid, biotin, pyridoxine, niacin, folic acid, diamino pentane, diamino hexane and dihydroxyacetone.

12. A method of producing biomass and/or an expression product in a cell culture, using a cell of claim 1 and a C₁ compound, preferably wherein the C₁ compound is the sole carbon source in the cell culture.

13. A fusion protein comprising a formolase (FLS) that is fused to a peroxisomal targeting signal (PTS), preferably wherein the fusion protein is characterized by one or more of the following features:
a) the PTS comprises or consists of an amino acid sequence selected from the group consisting of SKL, SEQ ID NO:17, 18, 19, 20, 21, 22, 23, 24, 25, and 26;
b) the FLS comprises of consists of SEQ ID NO:31 or SEQ ID NO:32, or a functional variant of any of the foregoing, preferably wherein
i) the functional variant of SEQ ID NO:31 comprises at least 1, 2, 3, 4, 5, 6, or 7 (all) of the point mutations A28L, W89R, R188H, N283H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:31;
ii) the functional variant of SEQ ID NO:32 comprises at least 1, 2, 3, 4, 5, 6, or 7 (all) of the point mutations A281, W89R, L90T, R188H, A394G, G419N, and A480W, preferably at least 3, 4, or 5 mutations selected from W89R, R188H, A394G, G419N, or A480W, point mutations as compared to SEQ ID NO:33, and at least at least any one of 90%, 95%, 96%, 97%, 98%, 99% or 100% sequence identity to SEQ ID NO:32.

14. A nucleic acid molecule encoding the fusion protein of claim 13, preferably wherein the nucleic acid molecule is codon-optimized for expression in a eukaryotic host cell.

15. An expression cassette comprising the nucleic acid molecule of claim 14 and regulatory sequences to express the fusion protein in a eukaryotic host cell.


SEQUENCE LISTING (44 sequences)

SEQ ID NO: 1 - DNA, 1764 bp, synthetic construct
  source 1..1764: mol_type = other DNA
atggcaatgatcacgggcggagaactggtagttagaacactcattaaggccggtgtggaacatttgtttggtctgcatggtctgcatatagacacaattttccaagcctgcttggaccacgatgttcctatcattgacactaggcacgaagccgccgctggacatgctgccgagggctatgcacgtgctggagctaaattaggcgttgccttagtcaccgccggaggagggttcacaaatgcagttactcccattgctaacgccagactagatcgaacacctgttttatttctaaccggcagtggtgcattgagagacgacgaaactaatacactgcaggccggaatcgaccaggtcgccatggccgcccctataactaaatgggcacatcgtgttatggcaacagagcatatcccgaggcttgttatgcaagcaatccgtgcagctctttcagcaccccgtggaccagtcttgctagacttaccatgggacattctcatgaatcaaatagatgaggactccgtgattataccagacctcgtactatccgcccacggtgctcatccagacccagctgacctcgaccaggccctagctctgcttagaaaggccgaacgtcctgtcatcgtactcggatcagaagctagtcgtactgcacgtaagactgcattaagcgcatttgtcgcagcaactggagtacccgtgttcgcagactacgaaggcctgtcaatgttgagcgggcttcctgatgctatgcgaggtggattggtccagaatctctattccttcgcaaaagctgacgctgctccggatttggtattgatgttgggtgcccgtttcggtctacatactggccacggatcaggacaactgatccctcattctgcacaggtcatccaagttgatcctgacgcttgtgagttgggacgtctacaggggattgctttaggaatcgtagccgatgttggtggtacaattgaggcacttgcccaggccaccgcacaagatgccgcttggcccgacagaggagattggtgcgcaaaagtgactgacttggctcaagaaagatatgcttcaatcgccgctaagagcagctcggagcatgctctgcaccccttccacgcatcccaagtaatcgcaaaacatgtcgacgccggtgtgaccgttgttgccgatggtggactaacctatttgtggctatcggaagttatgtcgagggttaagcctggcggcttcttatgccatggatacctaaattctatgggagtgggttttggaaccgcacttggagctcaggttgccgacctagaggcagggagacgtactatcctggtcaccggtgacgggtcggtggggtactcaatcggtgagttcgatacacttgtgcgtaaacagctgccgttaattgtgataataatgaacaaccaatcttggggatggacactacacttccagcagctagccgtaggaccaaaccgagttacaggtacaaggcttgaaaacggttcctatcacggggttgccgccgcctttggtgccgacggttaccacgttgactcagttgaaagtttctctgcagcccttgctcaggctctggcacataacaggcctgcctgcatcaatgtcgccgtagcattggaccccatcccaccagaggaattgatcttaatcggaatggatccattcgcaggctctaccgagaatttgtatttccaatcaggtgccttagggacgtacgaaaagactggcttagcccgattttaa

SEQ ID NO: 2 - protein, 587 aa, synthetic construct
MAMITGGELVVRTLIKAGVEHLFGLHGLHIDTIFQACLDHDVPIIDTRHEAAAGHAAEGYARAGAKLGVALVTAGGGFTNAVTPIANARLDRTPVLFLTGSGALRDDETNTLQAGIDQVAMAAPITKWAHRVMATEHIPRLVMQAIRAALSAPRGPVLLDLPWDILMNQIDEDSVIIPDLVLSAHGAHPDPADLDQALALLRKAERPVIVLGSEASRTARKTALSAFVAATGVPVFADYEGLSMLSGLPDAMRGGLVQNLYSFAKADAAPDLVLMLGARFGLHTGHGSGQLIPHSAQVIQVDPDACELGRLQGIALGIVADVGGTIEALAQATAQDAAWPDRGDWCAKVTDLAQERYASIAAKSSSEHALHPFHASQVIAKHVDAGVTVVADGGLTYLWLSEVMSRVKPGGFLCHGYLNSMGVGFGTALGAQVADLEAGRRTILVTGDGSVGYSIGEFDTLVRKQLPLIVIIMNNQSWGWTLHFQQLAVGPNRVTGTRLENGSYHGVAAAFGADGYHVDSVESFSAALAQALAHNRPACINVAVALDPIPPEELILIGMDPFAGSTENLYFQSGALGTYEKTGLARF

SEQ ID NO: 3 - DNA, 2987 bp, synthetic construct
  source 1..2987: mol_type = other DNA
caatctgtggtttgctaaactggaagtctggtaaggactctagcaagtccgttactcaaaaagtcataccaagtaagattacgtaacacctgggcatgactttctaagttagcaagtcaccaagagggtcctatttaacgtttggcggtatctgaaacacaagacttgcctatcccatagtacatcatattacctgtcaagctatgctaccccacagaaataccccaaaagttgaagtgaaaaaatgaaaattactggtaacttcaccccataacaaacttaataatttctgtagccaatgaaagtaaaccccattcaatgttccgagatttagtatacttgcccctataagaaacgaaggatttcagcttccttaccccatgaacagaaatcttccatttaccccccactggagagatccgcccaaacgaacagataatagaaaaaagaaattcggacaaatagaacactttctcagccaattaaagtcattccatgcactccctttagctgccgttccatccctttgttgagcaacaccatcgttagccagtacgaaagaggaaacttaaccgataccttggagaaatctaaggcgcgaatgagtttagcctagatatccttagtgaagggttgttccgatacttctccacattcagtcatagatgggcagctttgttatcatgaagagacggaaacgggcattaagggttaaccgccaaattatataaagacaacatgtccccagtttaaagtttttctttcctattcttgtatcctgagtgaccgttgtgtttaatataacaagttcgttttaacttaagaccaaaaccagttacaacaaattataacccctctaaacactaaagttcactcttatcaaactatcaaacatcaaaacatggcaatgatcacgggcggagaactggtagttagaacactcattaaggccggtgtggaacatttgtttggtctgcatggtctgcatatagacacaattttccaagcctgcttggaccacgatgttcctatcattgacactaggcacgaagccgccgctggacatgctgccgagggctatgcacgtgctggagctaaattaggcgttgccttagtcaccgccggaggagggttcacaaatgcagttactcccattgctaacgccagactagatcgaacacctgttttatttctaaccggcagtggtgcattgagagacgacgaaactaatacactgcaggccggaatcgaccaggtcgccatggccgcccctataactaaatgggcacatcgtgttatggcaacagagcatatcccgaggcttgttatgcaagcaatccgtgcagctctttcagcaccccgtggaccagtcttgctagacttaccatgggacattctcatgaatcaaatagatgaggactccgtgattataccagacctcgtactatccgcccacggtgctcatccagacccagctgacctcgaccaggccctagctctgcttagaaaggccgaacgtcctgtcatcgtactcggatcagaagctagtcgtactgcacgtaagactgcattaagcgcatttgtcgcagcaactggagtacccgtgttcgcagactacgaaggcctgtcaatgttgagcgggcttcctgatgctatgcgaggtggattggtccagaatctctattccttcgcaaaagctgacgctgctccggatttggtattgatgttgggtgcccgtttcggtctacatactggccacggatcaggacaactgatccctcattctgcacaggtcatccaagttgatcctgacgcttgtgagttgggacgtctacaggggattgctttaggaatcgtagccgatgttggtggtacaattgaggcacttgcccaggccaccgcacaagatgccgcttggcccgacagaggagattggtgcgcaaaagtgactgacttggctcaagaaagatatgcttcaatcgccgctaagagcagctcggagcatgctctgcaccccttccacgcatcccaagtaatcgcaaaacatgtcgacgccggtgtgaccgttgttgccgatggtggactaacctatttgtggctatcggaagttatgtcgagggttaagcctggcggcttcttatgccatggatacctaaattctatgggagtgggttttggaaccgcacttggagctcaggttgccgacctagaggcagggagacgtactatcctggtcaccggtgacgggtcggtggggtactcaatcggtgagttcgatacacttgtgcgtaaacagctgccgttaattgtgataataatgaacaaccaatcttggggatggacactacacttccagcagctagccgtaggaccaaaccgagttacaggtacaaggcttgaaaacggttcctatcacggggttgccgccgcctttggtgccgacggttaccacgttgactcagttgaaagtttctctgcagcccttgctcaggctctggcacataacaggcctgcctgcatcaatgtcgccgtagcattggaccccatcccaccagaggaattgatcttaatcggaatggatccattcgcaggctctaccgagaatttgtatttccaatcaggtgccttagggacgtacgaaaagactggcttagcccgattttaagcttcgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctgg

SEQ ID NO: 4 - DNA, 2895 bp, synthetic construct
  source 1..2895: mol_type = other DNA
agacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctactaggctactaacaccgtgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaaccatggcaatgatcacgggcggagaactggtagttagaacactcattaaggccggtgtggaacatttgtttggtctgcatggtctgcatatagacacaattttccaagcctgcttggaccacgatgttcctatcattgacactaggcacgaagccgccgctggacatgctgccgagggctatgcacgtgctggagctaaattaggcgttgccttagtcaccgccggaggagggttcacaaatgcagttactcccattgctaacgccagactagatcgaacacctgttttatttctaaccggcagtggtgcattgagagacgacgaaactaatacactgcaggccggaatcgaccaggtcgccatggccgcccctataactaaatgggcacatcgtgttatggcaacagagcatatcccgaggcttgttatgcaagcaatccgtgcagctctttcagcaccccgtggaccagtcttgctagacttaccatgggacattctcatgaatcaaatagatgaggactccgtgattataccagacctcgtactatccgcccacggtgctcatccagacccagctgacctcgaccaggccctagctctgcttagaaaggccgaacgtcctgtcatcgtactcggatcagaagctagtcgtactgcacgtaagactgcattaagcgcatttgtcgcagcaactggagtacccgtgttcgcagactacgaaggcctgtcaatgttgagcgggcttcctgatgctatgcgaggtggattggtccagaatctctattccttcgcaaaagctgacgctgctccggatttggtattgatgttgggtgcccgtttcggtctacatactggccacggatcaggacaactgatccctcattctgcacaggtcatccaagttgatcctgacgcttgtgagttgggacgtctacaggggattgctttaggaatcgtagccgatgttggtggtacaattgaggcacttgcccaggccaccgcacaagatgccgcttggcccgacagaggagattggtgcgcaaaagtgactgacttggctcaagaaagatatgcttcaatcgccgctaagagcagctcggagcatgctctgcaccccttccacgcatcccaagtaatcgcaaaacatgtcgacgccggtgtgaccgttgttgccgatggtggactaacctatttgtggctatcggaagttatgtcgagggttaagcctggcggcttcttatgccatggatacctaaattctatgggagtgggttttggaaccgcacttggagctcaggttgccgacctagaggcagggagacgtactatcctggtcaccggtgacgggtcggtggggtactcaatcggtgagttcgatacacttgtgcgtaaacagctgccgttaattgtgataataatgaacaaccaatcttggggatggacactacacttccagcagctagccgtaggaccaaaccgagttacaggtacaaggcttgaaaacggttcctatcacggggttgccgccgcctttggtgccgacggttaccacgttgactcagttgaaagtttctctgcagcccttgctcaggctctggcacataacaggcctgcctgcatcaatgtcgccgtagcattggaccccatcccaccagaggaattgatcttaatcggaatggatccattcgcaggctctaccgagaatttgtatttccaatcaggtgccttagggacgtacgaaaagactggcttagcccgattttaagctttcgatttgtatgtgaaatagctgaaattcgaaaatttcattatggctgtatctactttagcgtattaggcatttgagcattggcttgaacaatgcgggctgtagtgtgtcaccaaagaaaccattcgggttcggatctggaagtcctcatcacgtgatgccgatctcgtgtattttattttcagataacacctgaggacttt

SEQ ID NO: 5 - DNA, 23 bp, synthetic construct
  source 1..23: mol_type = other DNA
ggttagcctctaggcaaattctg

SEQ ID NO: 6 - DNA, 29 bp, synthetic construct
  source 1..29: mol_type = other DNA
gagtccctattctacttagtctcatatcg

SEQ ID NO: 7 - DNA, 24 bp, synthetic construct
  source 1..24: mol_type = other DNA
gcagctttgttatcatgaagagac

SEQ ID NO: 8 - DNA, 23 bp, synthetic construct
  source 1..23: mol_type = other DNA
ccttcctgttccatttattggtc

SEQ ID NO: 9 - DNA, 23 bp, synthetic construct
  source 1..23: mol_type = other DNA
ggaatactgctgatagcctaacg

SEQ ID NO: 10 - DNA, 24 bp, synthetic construct
  source 1..24: mol_type = other DNA
gtcataaccgagatagtctcacag

SEQ ID NO: 11 - DNA, 26 bp, synthetic construct
  source 1..26: mol_type = other DNA
gaagatcctttgatcttttctacggg

SEQ ID NO: 12 - DNA, 22 bp, synthetic construct
  source 1..22: mol_type = other DNA
cagtatagcgaccagcattcac

SEQ ID NO: 13 - DNA, 836 bp, synthetic construct
  source 1..836: mol_type = other DNA
ttaaacccataatnancgactatcggattgcaagtactagttttaaattgacaagattggggatgaactgtcgttatatatttgtcttccttcgacgggacagaatttggaaaaaaaaatagtagtaataatcgaatgaaagtattaacgctgaccaaggttaactgttcgaaaactaaaattgctgaaaattgctgttgaactcttctagttttttgttgattaataagctttgcgtacattatcactattatcactattatcactattcgaaagttctcctacagtcttacggtaaacggactctctacgtccgaagtaaaaactatgaaaaaataaacattggatatatcatatcctaaaaaaaacagtaaaacaaagaagagcatgctcgaacgaggactagtcggatagagcgtcgactacttatagaacaccatccccaaacccttttagtaagctcaaactacaaaaagaaccataaagggtgaggagaagtctcatgtcttctaattcactctgcaagcaaacacgttcgaagttgctacggttttcccatattattcgcagtaaacgtcgtaacacttcttttgatacaccgttcggttcggacgcttcttacataaaattcaaactgaaactacataagtgaactaattcggtattaagagctcatagatactaaccttcatacccttaccactatgggcgtaagaagtcacagaactccagaggatagtctaatacgggttgatttcgttggcctcctcctctaaagtaccatttaaagagactgaaaaccagtagtcatctgagcttgacactctgatagagcc

SEQ ID NO: 14 - DNA, 458 bp, synthetic construct
  source 1..458: mol_type = other DNA
acccaancggnagatccgtttaagacaagaagtatggatatagttgaaaagtagtctatcggaaacccaagttttttcttgatttcgtcctacggactatatatttagggtctactagacgaaaactttgataaaagtcatagaactaagcaaatgaatgtttgttgataacaactaaaatagacctcttattagcttgtttgtacattactattcctaggattcgaatgcccttcagaaatgtcaaaatcaatcctcgggaatatatactgtcattacgatcatgcaaaacaaaacaaattaattattgaatcaaatacaatcggatcatatctgaggtagttaaaaaaaacaataatgcattcggcgctactattatagactactttttaaggatagtcttttattaaatagttttcaaagtacgctatactctgattcatctattccctgaggtt

SEQ ID NO: 15 - DNA, 457 bp, synthetic construct
  source 1..457: mol_type = other DNA
cgtcgaaacaatagtacttctctgcctttgcccgtaattcccaattggcggtttaatatatttctgttgtacaggggtcaaatttcaaaaagaaaggataagaacataggactcactggcaacacaaattatattgttcaagcaaaattgaattctggttttggtcaatgttgtttaatattggggagatttgtgatttcaagtgagaatagtttgatagtttgtagttttgtacattactattcctaggattcgaacatctaaaccggtgattgcccaatcatcaacacattcagataatttaaactaaaaacaaatacctactagtagcatcaccgatagacaaatggacatcctgtaggatcccaccctaccactacatgtgggggagttagaagtctacgttgtgatacaccatccagtaactgtattccaaatcctttctggacaaaaaact

SEQ ID NO: 16 - protein, 578 aa, synthetic construct
MAMITGGELVVRTLIKAGVEHLFGLHGIHIDTIFQACLDHDVPIIDTRHEAAAGHAAEGYARAGAKLGVALVTAGGGFTNAVTPIANARTDRTPVLFLTGSGALRDDETNTLQAGIDQVAMAAPITKWAHRVMATEHIPRLVMQAIRAALSAPRGPVLLDLPWDILMNQIDEDSVIIPDLVLSAHGAHPDPADLDQALALLRKAERPVIVLGSEASRTARKTALSAFVAATGVPVFADYEGLSMLSGLPDAMRGGLVQNLYSFAKADAAPDLVLMLGARFGLNTGHGSGQLIPHSAQVIQVDPDACELGRLQGIALGIVADVGGTIEALAQATAQDAAWPDRGDWCAKVTDLAQERYASIAAKSSSEHALHPFHASQVIAKHVDAGVTVVADGGLTYLWLSEVMSRVKPGGFLCHGYLNSMGVGFGTALGAQVADLEAGRRTILVTGDGSVGYSIGEFDTLVRKQLPLIVIIMNNQSWGWTLHFQQLAVGPNRVTGTRLENGSYHGVAAAFGADGYHVDSVESFSAALAQALAHNRPACINVAVALDPIPPEELILIGMDPFAGSTENLYFQSGASKL

SEQ ID NO: 17-18 - skipped (2 entries)

SEQ ID NO: 19 - protein, 12 aa, Pichia pastoris
LGTYEKTGLARF

SEQ ID NO: 20 - protein, 12 aa, Pichia pastoris
HAAGTFKSESKL

SEQ ID NO: 21 - protein, 12 aa, Pichia pastoris
ITSLSVSVPARL

SEQ ID NO: 22 - protein, 12 aa, Pichia pastoris
VPSLFRRVLSKL

SEQ ID NO: 23 - protein, 12 aa, Pichia pastoris
QLSPRGDSAARL

SEQ ID NO: 24 - protein, 12 aa, Pichia pastoris
KHSSADRVLAKL

SEQ ID NO: 25 - protein, 9 aa, Saccharomyces cerevisiae
RLQSIKDHL

SEQ ID NO: 26 - protein, 9 aa, Hansenula polymorpha
RLRQIASQA

SEQ ID NO: 27 - protein, 663 aa, Komagataella phaffii
MAIPEEFDILVLGGGSSGSCIAGRLANLDHSLKVGLIEAGENNLNNPWVYLPGIYPRNMKLDSKTASFYTSNPSPHLNGRRAIVPCANVLGGGSSINFMMYTRGSASDYDDFQAEGWKTKDLLPLMKKTETYQRACNNPDIHGFEGPIKVSFGNYTYPVCQDFLRASESQGIPYVDDLEDLVTAHGAEHWLKWINRDTGRRSDSAHAFVHSTMRNHDNLYLICNTKVDKIIVEDGRAAAVRTVPSKPLNPKKPSHKIYRARKQIVLSCGTISSPLVLQRSGFGDPIKLRAAGVKPLVNLPGVGRNFQDHYCFFSPYRIKPQYESFDDFVRGDAEIQKRVFDQWYANGTGPLATNGIEAGVKIRPTPEELSQMDESFQEGYREYFEDKPDKPVMHYSIIAGFFGDHTKIPPGKYMTMFHFLEYPFSRGSIHITSPDPYAAPDFDPGFMNDERDMAPMVWAYKKSRETARRMDHFAGEVTSHHPLFPYSSEARALEMDLETSNAYGGPLNLSAGLAHGSWTQPLKKPTAKNEGHVTSNQVELHPDIEYDEEDDKAIENYIREHTETTWHCLGTCSIGPREGSKIVKWGGVLDHRSNVYGVKGLKVGDLSVCPDNVGCNTYTTALLIGEKTATLVGEDLGYSGEALDMTVPQFKLGTYEKTGLARF

SEQ ID NO: 28 - protein, 663 aa, Komagataella phaffii
MAIPEEFDILVLGGGSSGSCIAGRLANLDHSLKVGLIEAGENNLNNPWVYLPGIYPRNMKLDSKTASFYTSNPSPHLNGRRAIVPCANILGGGSSINFMMYTRGSASDYDDFEAEGWKTKDLLPLMKKTETYQRACNNPEIHGFEGPIKVSFGNYTYPVCQDFLRATESQGIPYVDDLEDLVTAHGAEHWLKWINRDTGRRSDSAHAFVHSTMRNHDNLYLICNTKVDKIIVEDGRAAAVRTVPSKPLNAKKPTHKVYRARKQIVLSCGTISSPLVLQRSGFGDPIKLRAAGVKPLVNLPGVGRNFQDHYCFFSPYRIKPQYESFDDFVRGDANIQKKVFDQWYANGTGPLATNGIEAGVKIRPTPEELSQMDESFQEGYREYFEDKPDKPVMHYSIIAGFFGDHTKIPPGKYMTMFHFLEYPFSRGSIHITSPDPYATPDFDPGFMNDERDMAPMVWSYKKSRETARKMDHFAGEVTSHHPLFPYSSEARAYEMDLETSNAYGGPLNLTAGLAHGSWTQPLKKPAGRNEGHVTSNQVELHPDIEYDEEDDKAIENYIREHTETTWHCLGTCSIGPREGSKIVKWGGVLDHRSNVYGVKGLKVGDLSVCPDNVGCNTYTTALLIGEKTATLVGEDLGYTGEALDMTVPQFKLGTYEKTGLARF

SEQ ID NO: 29 - protein, 663 aa, Candida boidinii
MAIPEEFDVIVCGGGSTGCVIAGRLANVDENLKVLLIENGENNLNNPWVYLPGIYPRNMRLDSKTATFYNSRPSKHLNGRRAIVPQANILGGGSSINFMMYTRASASDYDDWESEGWTTDELLPLMKKFETYQRPCNNRDVHGFDGPIKVSFGNYTYPQCQDFLRACETQGIPYVDDLEDLKTSHGAEQWLKWINRDFGRRSDTAHAFIHSTMRNKENLFLMTNTKVDKVIIEDGRAVAVRTVPSKPIGDSKVSRTFKARKQIVVSCGTVSSPMVLQRSGIGEPSKLRAAGVKPIVELPGVGRNFQDHFCYFVPYRIKQDSESFDAFVSGDKEAQKSAFDQWYATGAGPLATNGIEAGVKIRPTEAELATADKAFQQGWESYFENKPDKPLMHYSVISGFFGDHTRLPPGKYMTMFHFLEYPFSRGWLHISSDDPYAAPDFDPGFMNDDRDMWPMVWAFKKSRETARRMECFAGEPTAFHPHYKVDSPARALEQSAEDTKKVAGPLHLTANLYHGSWSTPIGEADKHDPNHVTSSHINVYSKDIQYTKEDDEAIENYIKEHAETTWHCLGTNSMAPREGNKNAPEGGVLDPRLNVHGVKGLKVADLSVCPDNVGCNTFSTALTIGEKAAVLVAEDLGYSGSELDMEVPQHKLKTYEQTGAARY

SEQ ID NO: 30 - protein, 664 aa, Hansenula polymorpha
MAIPDEFDIIVVGGGSTGCCIAGRLANLDDQNLTVALIEGGENNINNPWVYLPGVYPRNMRLDSKTATFYSSRPSKALNGRRAIVPCANILGGGSSINFLMYTRASASDYDDWESEGWSTDELLPLIKKIETYQRPCNNRDLHGFDGPIKVSFGNYTYPTCQDFLRAAESQGIPVVDDLEDFKTSHGAEHWLKWINRDLGRRSDSAHAYVHPTMRNKQSLFLITSTKCDKVIIEDGKAVAVRTVPMKPLNPKKPVSRTFRARKQIVISCGTISSPLVLQRSGIGAAHHLRSVGVKPIVDLPGVGENFQDHYCFFTPYYVKPDVPTFDDFVRGDPVAQKAAFDQWYSNKDGPLTTNGIEAGVKIRPTEEELATADEDFRRGYAEYFENKPDKPLMHYSVISGFFGDHTKIPNGKFMTMFHFLEYPFSRGFVRITSANPYDAPDFDPGFLNDERDLWPMVWAYKKSRETARRMESFAGEVTSHHPLFKVDSPARARDLDLETCSAYAGPKHLTANLYHGSWTVPIDKPTPKNDFHVTSNQVQLHSDIEYTEEDDEAIVNYIKEHTETTWHCLGTCSMAPREGSKIAPKGGVLDARLNVYGVQNLKVADLSVCPDNVGCNTYSTALTIGEKAATLVAEDLGYSGSDLDMTIPNFRLGTYEETGLARF

SEQ ID NO: 31 - protein, 575 aa, synthetic construct
MAMITGGELVVRTLIKAGVEHLFGLHGLHIDTIFQACLDHDVPIIDTRHEAAAGHAAEGYARAGAKLGVALVTAGGGFTNAVTPIANARLDRTPVLFLTGSGALRDDETNTLQAGIDQVAMAAPITKWAHRVMATEHIPRLVMQAIRAALSAPRGPVLLDLPWDILMNQIDEDSVIIPDLVLSAHGAHPDPADLDQALALLRKAERPVIVLGSEASRTARKTALSAFVAATGVPVFADYEGLSMLSGLPDAMRGGLVQNLYSFAKADAAPDLVLMLGARFGLHTGHGSGQLIPHSAQVIQVDPDACELGRLQGIALGIVADVGGTIEALAQATAQDAAWPDRGDWCAKVTDLAQERYASIAAKSSSEHALHPFHASQVIAKHVDAGVTVVADGGLTYLWLSEVMSRVKPGGFLCHGYLNSMGVGFGTALGAQVADLEAGRRTILVTGDGSVGYSIGEFDTLVRKQLPLIVIIMNNQSWGWTLHFQQLAVGPNRVTGTRLENGSYHGVAAAFGADGYHVDSVESFSAALAQALAHNRPACINVAVALDPIPPEELILIGMDPFAGSTENLYFQSGA

SEQ ID NO: 32 - protein, 575 aa, synthetic construct
MAMITGGELVVRTLIKAGVEHLFGLHGIHIDTIFQACLDHDVPIIDTRHEAAAGHAAEGYARAGAKLGVALVTAGGGFTNAVTPIANARTDRTPVLFLTGSGALRDDETNTLQAGIDQVAMAAPITKWAHRVMATEHIPRLVMQAIRAALSAPRGPVLLDLPWDILMNQIDEDSVIIPDLVLSAHGAHPDPADLDQALALLRKAERPVIVLGSEASRTARKTALSAFVAATGVPVFADYEGLSMLSGLPDAMRGGLVQNLYSFAKADAAPDLVLMLGARFGLNTGHGSGQLIPHSAQVIQVDPDACELGRLQGIALGIVADVGGTIEALAQATAQDAAWPDRGDWCAKVTDLAQERYASIAAKSSSEHALHPFHASQVIAKHVDAGVTVVADGGLTYLWLSEVMSRVKPGGFLCHGYLNSMGVGFGTALGAQVADLEAGRRTILVTGDGSVGYSIGEFDTLVRKQLPLIVIIMNNQSWGWTLHFQQLAVGPNRVTGTRLENGSYHGVAAAFGADGYHVDSVESFSAALAQALAHNRPACINVAVALDPIPPEELILIGMDPFAGSTENLYFQSGA

SEQ ID NO: 33 - protein, 563 aa, Pseudomonas fluorescens
MAMITGGELVVRTLIKAGVEHLFGLHGAHIDTIFQACLDHDVPIIDTRHEAAAGHAAEGYARAGAKLGVALVTAGGGFTNAVTPIANAWLDRTPVLFLTGSGALRDDETNTLQAGIDQVAMAAPITKWAHRVMATEHIPRLVMQAIRAALSAPRGPVLLDLPWDILMNQIDEDSVIIPDLVLSAHGARPDPADLDQALALLRKAERPVIVLGSEASRTARKTALSAFVAATGVPVFADYEGLSMLSGLPDAMRGGLVQNLYSFAKADAAPDLVLMLGARFGLNTGHGSGQLIPHSAQVIQVDPDACELGRLQGIALGIVADVGGTIEALAQATAQDAAWPDRGDWCAKVTDLAQERYASIAAKSSSEHALHPFHASQVIAKHVDAGVTVVADGALTYLWLSEVMSRVKPGGFLCHGYLGSMGVGFGTALGAQVADLEAGRRTILVTGDGSVGYSIGEFDTLVRKQLPLIVIIMNNQSWGATLHFQQLAVGPNRVTGTRLENGSYHGVAAAFGADGYHVDSVESFSAALAQALAHNRPACINVAVALDPIPPEELILIGMDPFA

SEQ ID NO: 34 - protein, 12 aa, synthetic construct
GSTENLYFQSGA

SEQ ID NO: 35-44 - skipped (10 entries)